(19) United States
(12) Patent Application Publication
(10) Pub. No.: US 2026/0115500 A1
(43) Pub. Date: Apr. 30, 2026

(54) METHODS AND SYSTEMS FOR HISTOTRIPSY

(71) Applicant: SCITON, INC., Palo Alto, CA (US)

(72) Inventors: Charles D. EMERY, Palo Alto, CA (US); Daniel K. NEGUS, Palo Alto, CA (US); James L. HOBART, Palo Alto, CA (US); Thomas Craig BRINTON, Palo Alto, CA (US)

(73) Assignee: SCITON, INC., Palo Alto, CA (US)

(21) Appl. No.: 19/151,566

(22) PCT Filed: Feb. 2, 2024
(86) PCT No.: PCT/US2024/014227

§ 371 Date: Jul. 28, 2025

Related U.S. Application Data
(60) Provisional application No. 63/443,209, filed on Feb. 3, 2023.

(51) Int. Cl. A61N 7/02; A61N 7/00

(52) U.S. Cl. CPC A61N 7/02; A61N 2007/0039; A61N 2007/0052

(57) ABSTRACT

Methods, systems, and computer storage media for the improved treatment of biological tissue, such as skin, muscle, fat, nerves, or glands, or components thereof, are described herein. In some embodiments, a method of treating a patient's biological tissue comprises positioning an ultrasound pressure wave in a target volume of the region of biological tissue to form a cavitation bubble cloud in the target volume using the pressure wave, and maintaining the cavitation bubble cloud in the target volume for a time period sufficient to produce within the target volume. In some embodiments, the homogenate is fractionated within non-homogenized or healthy biological tissue in the region of biological tissue.

### RELATED APPLICATION DATA

This application claims priority pursuant to 35 U.S.C. § 119(e) to U.S. Provisional Patent Application Ser. No. 63/443,209, filed on Feb. 3, 2023, which is incorporated herein by reference in its entirety.

### FIELD

The technology described herein generally relates to the treatment of biological tissue (e.g., skin, muscle, fat, nerves, and/or glands), and more specifically to treatment of tissue using ultrasound.

### BACKGROUND

Histotripsy refers to the application of acoustic energy (such as provided by a focused ultrasound beam) to a target volume inside the body of a patient. Histotripsy can be used to ablate biological tissue, particularly by initiating, maintaining, and controlling acoustic cavitation within a desired location within the body. Such histotripsy ablation of tissue generally occurs through “homogenization” or “fractionation” of tissue, which means the tissue is broken down into a suspension of cell fragments and cell constituents using acoustic energy.

Current systems and methods for histotripsy can suffer from one or more disadvantages, including for clinical applications. For example, some systems and methods for histotripsy can present safety concerns or have low efficiency. Accordingly, there exists a therapeutic need for improved systems and methods of treatment using histotripsy that can provide both efficacy and reduction in risk of adverse events, such as overtreatment or undertreatment of target tissue and/or damage to healthy tissue.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Embodiments of the technology described herein are directed towards treatment of biological tissue, for instance that of a patient, that realizes improved safety and clinical results. The disclosed technology can be used to treat various types of biological tissue of a patient (such as a human or animal patient) in need thereof, including, for instance, skin tissue, muscle tissue, fat tissue, nerve tissue, cartilage, other connective tissue (e.g., fibrous septae), and/or glands, and/or components of skin, muscle, fat, nerves, glands, and/or other tissue on or within a patient. In some non-limiting cases, for instance, the disclosed technology can be used to treat individual nerves or skin glands such as sudoriferous glands, sebaceous glands, and/or hair follicles.

In general, methods, systems, and computer storage media for the improved treatment of biological tissue (e.g., skin, muscle, fat, nerves, cartilage, connective tissue, and/or glands, or a component thereof) are described herein. In some embodiments, a method of treating a patient's biological tissue (e.g., skin, muscle, fat, nerves, cartilage, connective tissue and/or glands), and/or a component thereof, comprises positioning an ultrasound pressure wave in a target volume of the region of biological tissue to form a cavitation bubble cloud in the target volume using the pressure wave, and maintaining the cavitation bubble cloud in the target volume for a time period sufficient to produce homogenate or to homogenize tissue, or to produce homogenized tissue within the target volume, such as, for instance, through the repeated expansion and contraction of the bubbles.

Moreover, the foregoing process can be repeated a number of times to treat a region of biological tissue. That is, multiple target volumes differing from one another can be treated with an ultrasound pressure wave, sequentially or in parallel. In some embodiments, for example, a method described herein further comprises positioning a pressure wave in a second target volume of a region of biological tissue to form a second cavitation bubble cloud in the second target volume. The second cavitation bubble cloud can be maintained in the second target volume for a second time period sufficient to produce homogenate or to homogenize tissue or to produce homogenized tissue within the second target volume. As described above, the second target volume can differ from the first target volume in such embodiments, and additional target volumes may also be treated. Thus, in some cases, a method described herein further comprises positioning the pressure wave in n additional target volumes of the region of biological tissue to form n additional cavitation bubble clouds in the n additional target volumes, and maintaining the n additional cavitation bubble clouds in the n additional target volumes for n additional time periods sufficient to produce homogenate or to homogenize tissue, or to produce homogenized tissue within the n additional target volumes. The n additional target volumes differ from one another and from the first target volume and the second target volume, and n can be any integer not inconsistent with the technical objectives of the present disclosure, such as an integer from 1 to 1,000,000. It is further to be understood that each target volume can denote or correspond to a different treatment location, and, in some cases, the ultrasound beam/pressure wave can be moved from one location to another as the method of treatment is carried out, such as, for example, by using mechanical translation, mechanical rotation, electronic steering and focusing, or any combination thereof, of the ultrasound beam/pressure wave or the source thereof, relative to the patient or treatment area.

Methods described herein, in some cases, can also comprise or be characterized by certain additional steps or features (or a combination of additional steps and features) that can provide one or more advantages compared to other methods. For example, in some embodiments, the homogenate or homogenized tissue (of a first, second, and/or nth target volume) is fractionated within non-homogenized or healthy biological tissue. In still other embodiments, a pressure wave of a method described herein is a composite pressure wave comprising a high frequency component wave and a low frequency component wave.

In some embodiments, at least one cycle of the high frequency component wave is positioned in a trough of at least one cycle of the low frequency component wave. For instance, in some embodiments, such positioning may increase a likelihood of reaching a target cavitation pressure without requiring significant thermal energy.

It is also possible for the high frequency component wave to be disposed in a compressional part of the low frequency component wave. For instance, in some embodiments, this may be used when or after cavitation is created. In some embodiments, disposing the high frequency component wave in the compressional part of the low frequency wave may decrease the amplitude required to maintain a bubble cloud. In some embodiments, disposing the high frequency component wave in the compressional part of the low frequency wave may increase the chance of shock scattering. In some embodiments, disposing the high frequency component wave in the compressional part of the low frequency wave may increase a propensity to create harmonics, thus creating a more nonlinear heating at the intended treatment region.

Further, in some instances of a method described herein, maintaining a cavitation bubble cloud in a target volume does not deliver a thermal dose to tissue within the target volume that is sufficient to create denaturation, coagulation, or apoptosis. Alternatively, in other embodiments, maintaining a cavitation bubble cloud in a target volume does deliver a thermal dose to tissue within the target volume that is sufficient to create denaturation, coagulation, or apoptosis.

Moreover, in some embodiments, positioning a pressure wave in a target volume comprises applying an ultrasound beam to the target volume, wherein the ultrasound beam may be a pulsed ultrasound beam. Further, a focus of the ultrasound beam can be positioned in the target volume. Additionally, in some cases, the focus of the ultrasound beam is a point focus. In other instances, the focus of the ultrasound beam is a line focus. In still other embodiments, the focus comprises or consists of a combination of simultaneous lines and point foci or a combination of simultaneous multiple lines or point foci.

Further, in some embodiments, a method as described herein further comprises detecting the onset of cavitation, which is the time t=0 when the cavitation bubble cloud in the target volume begins to form. In some embodiments, the onset of cavitation may be detected based on acoustic cavitation emissions. In some embodiments, a transducer is used as a detection device. In some cases, the same transducer used to transmit an ultrasound pulse may also be used as a detection device. In other embodiments, a transducer used as the detection device (e.g., to detect acoustic cavitation emissions) is different from a transducer employed to apply an ultrasound pulse. In some cases, a transducer may be used to transmit another ultrasound pulse toward the region where the cavitation may be occurring and detect the backscatter off of this region as a means to determine the presence of cavitation. This transducer may also be the same as or different from the therapy transducer.

Systems for treating a region of biological tissue (such as skin, muscle, fat, nerves, cartilage, connective tissue, and/or glands) are also described herein. In some embodiments, a system for treating biological tissue of a patient comprises one or more ultrasound transducers to provide an ultrasound pressure wave to a target volume in the region of biological tissue and to form a cavitation bubble cloud in the target volume. In some cases, the system may include a tissue acquisition system (e.g., a vacuum) which may contain one or more transducers that create an ultrasound pressure wave within the acquired tissue volume and generate a cavitation bubble cloud as described herein. The system can also comprise a control device to maintain the cavitation bubble cloud in the target volume for a time period sufficient to produce homogenate within the target volume. In addition, in some cases, a system described herein can comprise one or more sources of electromagnetic radiation to provide one or more beams of electromagnetic radiation to the region of biological tissue for imaging, thermal ablation, and/or delivering non-ablating energy to the region of biological tissue. Moreover, a system according to the present disclosure can also comprise, in some embodiments, one or more microneedles to provide treatment to skin. In some embodiments, a system according to the present invention may be a “hybrid” system, comprising multiple features as described above (e.g., one or more microneedle(s), detection transducer(s), therapy transducer(s), additional ultrasound components, such as additional transducer(s), tissue acquisition system(s), control device(s), source(s) of radiation, such as electromagnetic radiation, and/or broadband light therapy system(s)).

In still another aspect, improved computer devices and storage media are also described herein. For example, computer storage media storing computer-useable instructions are described herein. Such a computer storage medium can be used to carry out a method and/or use a system described herein. For instance, in some cases, a computer storage medium stores computer-useable instructions that, when used by one or more computing devices, cause the one or more computing devices to treat a region of biological tissue of a patient, the operations comprising: positioning an ultrasound pressure wave in a target volume of the region of biological tissue to form a cavitation bubble cloud in the target volume; and maintaining the cavitation bubble cloud in the target volume for a time period sufficient to produce homogenate within the target volume, wherein the homogenate is fractionated within non-homogenized biological tissue in the region of biological tissue. In some instances, the operations further comprise forming a thermal coagulation zone around the target volume. In still other cases, the operations further comprise positioning the pressure wave in n additional target volumes of the region of biological tissue to form n additional cavitation bubble clouds in the n additional target volumes; and maintaining the n additional cavitation bubble clouds in the n additional target volume for n additional time periods sufficient to produce homogenate within the n additional target volumes, wherein the n additional target volumes differ from one another and from the first target volume, and wherein n is an integer from 1 to 1,000,000.

Additional objects, advantages, and novel features of the invention will be set forth in part in the description which follows, and in part will become apparent to those skilled in the art upon examination of the following, or can be learned by practice of the invention.

### BRIEF DESCRIPTION OF THE DRAWINGS

Aspects of the technology presented herein are described in detail below with reference to the accompanying drawing figures, which are not necessarily drawn to scale.

FIG. 1A illustrates a pattern of homogenized regions produced by one embodiment of a method described herein.

FIG. 1B illustrates a pattern of homogenized regions produced by one embodiment of a method described herein.

FIG. 2A illustrates a pattern of homogenized regions produced by one embodiment of a method described herein.

FIG. 2B illustrates a pattern of homogenized regions produced by one embodiment of a method described herein.

FIG. 3A illustrates a pattern of homogenized regions produced by one embodiment of a method described herein.

FIG. 3B illustrates a pattern of homogenized regions produced by one embodiment of a method described herein.

FIG. 4A illustrates a pattern of homogenized regions produced by one embodiment of a method described herein.

FIG. 4B illustrates a pattern of homogenized regions produced by one embodiment of a method described herein.

FIG. 5A illustrates a composite pressure wave according to one embodiment described herein.

FIG. 5B illustrates an enlarged portion of FIG. 5A.

FIG. 6A illustrates a composite pressure wave according to one embodiment described herein.

FIG. 6B illustrates an enlarged portion of FIG. 6A.

FIG. 7 illustrates a top view of a composite ultrasound transducer or array in accordance with one embodiment described herein.

FIG. 8 illustrates a top view of a first target volume and a second target volume treated in accordance with one embodiment described herein.

FIG. 9A illustrates a system according to one embodiment described herein.

FIG. 9B illustrates a system according to another embodiment described herein.

FIG. 10 illustrates steps of a method according to one embodiment described herein.

FIG. 11 illustrates a block diagram of an example computing environment and/or device architecture in which some implementations of the present technology may be employed.

FIG. 12 illustrates a cross-section of a bowl-shaped ultrasound transducer.

FIG. 13A illustrates a perspective view of an ultrasound transducer according to one embodiment described herein.

FIG. 13B illustrates a sectional view of an ultrasound transducer according to one embodiment described herein.

FIG. 14A illustrates a cylinder corresponding to an ultrasound transducer according to one embodiment described herein.

FIG. 14B illustrates a cylinder corresponding to an ultrasound transducer according to one embodiment described herein.

FIG. 14C illustrates a perspective view of an ultrasound transducer according to one embodiment described herein.

FIG. 14D illustrates a sectional view of an ultrasound transducer according to one embodiment described herein.

FIG. 14E illustrates a perspective view of an ultrasound transducer according to one embodiment described herein.

FIG. 15 illustrates a sectional view of an ultrasound transducer according to one embodiment described herein.

FIG. 16 illustrates a sectional view of an ultrasound transducer according to one embodiment described herein.

FIG. 17 illustrates a perspective view of an ultrasound transducer according to one embodiment described herein.

FIG. 18 illustrates a perspective view of a reflector of a device according to one embodiment described herein.

FIG. 19 illustrates a perspective view of a device according to one embodiment described herein.

FIG. 20 illustrates a perspective view of a device according to one embodiment described herein.

FIG. 21 illustrates a perspective view of a device according to one embodiment described herein.

FIG. 22 illustrates a perspective view of a device according to one embodiment described herein.

FIG. 23 illustrates a perspective view of a device according to one embodiment described herein.

FIG. 24 illustrates various transducer designs according to embodiments described herein.

### DETAILED DESCRIPTION

The subject matter of aspects of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps disclosed herein unless and except when the order of individual steps is explicitly described.

Accordingly, embodiments described herein can be understood more readily by reference to the following detailed description, examples, and figures. Elements, features, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. All ranges disclosed herein are also to be considered to include the end points of the range, unless expressly stated otherwise. For example, a range of “between 5 and 10” or “5 to 10” or “5-10” should generally be considered to include the end points 5 and 10, as well as any and all subranges falling within these values.

Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

Additionally, in any disclosed embodiment, the terms “substantially,” “approximately,” and “about” may be substituted with “within [a percentage] of” what is specified, where the percentage includes 0.1, 1, 5, and 10 percent.

It is also to be understood that the article “a” or “an” refers to “at least one,” unless the context of a particular use requires otherwise.

In one aspect, methods of treating a human or animal patient or biological tissue of a human or animal patient are described herein. In some embodiments, such a method comprises positioning an ultrasound pressure wave in a target volume of the region of biological tissue to form a cavitation bubble cloud in the target volume using the pressure wave, and maintaining the cavitation bubble cloud in the target volume for a time period sufficient to produce homogenate or to homogenize tissue, or to produce homogenized tissue within the target volume. Moreover, the foregoing process can be repeated a desired number of times to treat a region of biological tissue. That is, multiple target volumes differing from one another can be treated with an ultrasound pressure wave as described herein. In some embodiments, for example, a method described herein further comprises positioning the pressure wave in a second target volume of the region of biological tissue to form a second cavitation bubble cloud in the second target volume. The second cavitation bubble cloud can be maintained in the second target volume for a second time period sufficient to produce homogenate or to homogenize tissue, or to produce homogenized tissue within the second target volume. As described above, the second target volume can differ from the first target volume in such embodiments, and additional target volumes may also be treated. Thus, in some cases, a method described herein further comprises positioning the pressure wave in n additional target volumes of the region of biological tissue to form n additional cavitation bubble clouds in the n additional target volumes, and maintaining the n additional cavitation bubble clouds in the n additional target volumes for n additional time periods sufficient to produce homogenate or to homogenize tissue, or to produce homogenized tissue within the n additional target volumes. The n additional target volumes differ from one another and from the first target volume and the second target volume, and n can be any integer not inconsistent with the technical objectives of the present disclosure, such as an integer from 1 to 1,000,000. It is further to be understood that each target volume can denote or correspond to a different treatment location, and, in some cases, the ultrasound beam/pressure wave can be moved from one location to another as the method of treatment is carried out.

For clarity and convenience, when a target volume (or tissue thereof) or a plurality of target volumes (or tissue thereof) is described in the present disclosure, it is to be understood that the target volume can be a first, second, or nth target volume, unless the context requires otherwise. Additionally, as described further herein, one or more target volumes treated by a method described herein can collectively define a treatment pattern or can collectively provide a single treatment of the relevant region of biological tissue.

A pressure wave can be positioned in a target volume (or a plurality or series of target volumes) in any manner not inconsistent with the technical objectives of the present disclosure. For example, in some implementations, positioning a pressure wave in a target volume comprises applying an ultrasound beam to the target volume. The ultrasound beam can have any properties and be provided in any manner not inconsistent with the technical objectives of the present disclosure. In some cases, for instance, the ultrasound beam is a pulsed ultrasound beam. Such a beam, in some instances, can be provided by one or more ultrasound transducers, such as one or more high intensity focused ultrasound (HIFU) transducers. In addition, in some embodiments described herein, a focus of one or more ultrasound beams is positioned in a target volume (or a plurality or series of target volumes). Moreover, the focus of an ultrasound beam described herein can have any size and shape not inconsistent with the technical objectives of the present disclosure. In some cases, for example, the focus of an ultrasound beam is a point focus (i.e., a focus in three dimensions, such as x, y, and z). The point focus may be symmetric or asymmetric in x, y, and/or z. In other words, the region size within the ‘focus’ where the pressure exceeds the necessary cavitational threshold may vary in x, y, and z.

In other instances, as described further hereinbelow in the Examples, the focus of an ultrasound beam is a line focus or a focus in two dimensions (e.g., in x,z or y,z). In this instance, the ultrasound beam can have at least one dimension which is not either mechanically or electronically focused. It is also possible for a focus of an ultrasound beam described herein to be a compound focus, such as a focus in both x,z and y,z, where the depth (z) of the focus for the two foci can vary. Such a configuration can enable the creation of a line focus where the focal gain is slightly higher than a design with a focus in only x,z or y,z, and can allow cavitational thresholds to be exceeded. It is further to be understood that a focus described herein can be described in one or more dimensions using Cartesian coordinates (x,y,z) or other coordinate systems, such as spherical coordinates (rho, theta, phi) or cylindrical coordinates (rho, theta, z), and the coordinate system is not particularly limited.

In some embodiments, a method described herein can include a step of detecting the onset of cavitation, as described in more detail hereinbelow. Cavitation onset detection can allow a user to select or optimize one or more system parameters. The onset of cavitation is defined as the time t=0 when the cavitation bubble cloud in the target volume begins to form. The point at which bubbles begin to form (e.g., at the time t=0) can be detected in any manner not inconsistent with the technical objectives of the present disclosure. For example, in some cases, the onset of cavitation is detected based on acoustic cavitation emissions (akin to “listening” for bubbles). Any detection device not inconsistent with the objectives of this invention may be employed to detect acoustic cavitation emissions and/or to detect the onset of cavitation. In some embodiments, a transducer can be used as a detection device to detect the onset of cavitation and/or to detect acoustic cavitation emissions. In some embodiments, the same transducer used to apply an ultrasound pulse to a target volume is also used to detect acoustic cavitation emissions and/or to detect the onset of cavitation. In other embodiments, a transducer used as the detection device (e.g., to detect acoustic cavitation emissions) is different from a transducer employed to apply an ultrasound pulse. In some cases, a transducer may be used to transmit another ultrasound pulse toward the region where the cavitation may be occurring and detect the backscatter off of this region as a means to determine the presence of cavitation. This transducer may be the same as or different from the therapy transducer. In some embodiments, the onset of cavitation can be detected simultaneously with emission of an interrogation pulse. In some embodiments, a detection device can also be used at other points of the process to determine cavitation progress. That is, in some embodiments, a method described herein comprises applying an ultrasound pulse or an interrogation pulse to a target volume while simultaneously detecting acoustic cavitation emissions at the onset of cavitation and/or throughout treatment.

It is also possible for a method or system described herein to use more than one ultrasound beam and/or more than one ultrasound transducer or other ultrasound source. For example, in some embodiments, a plurality of ultrasound beams is used to provide a single focus or an overlapping focus region. In some such cases, for instance, multiple ultrasound beams are positioned such that the respective foci of the ultrasound beams overlap and/or have an interference pattern that provides a “combined” focus that is different in size, shape, and/or peak negative pressure compared to the focus of any individual ultrasound beam that is used to form the “combined” focus. In some cases, these techniques can further increase the achievable pressure and/or modify the shape of the pressure field at the intended focus such that the relevant cavitational threshold is exceeded.

Further, in some cases, a pressure wave (or a “combined” focus of a combination of pressure waves) of a method described herein has a peak negative pressure of 10 MPa to 100 MPa, 10 MPa to 80 MPa, 10 MPa to 75 MPa, 10 MPa to 50 MPa, 20 MPa to 100 MPa, 20 MPa to 80 MPa, 20 MPa to 50 MPa, or 20 MPa to 40 MPa. It is further to be understood that the foregoing peak negative pressure can be achieved through coherent summation of the pressure wave(s) or reflection off of the bubble cloud, reflection off of a bone, and/or reflection off of a coagulated region, as described further herein. Moreover, in some embodiments, the peak negative pressure of the pressure wave used in a method described herein is selected based on the target tissue type. For example, in some instances, the peak negative pressure can be selected in accordance with the data in Table 1 below, obtained from Vlaisavljevich et. al., “Histotripsy-Induced Cavitation Cloud Initiation Thresholds in Tissues of Different Mechanical Properties,” IEEE Transactions on Ultrasonics, Ferroelectrics, and Frequency Control, Vol. 61, No. 2, February 2014 (hereinafter “Vlaisavljevich”).

TABLE 1
Minimum Peak Negative Pressures for Various Tissue Types

| Tissue Type | Minimum Peak Negative Pressure (MPa) |
|---|---|
| Fat | 13.26 |
| Muscle | 19.12 |
| Skin | 23.21 |
| Tendon | 24.47 |

Moreover, a method described herein can be used to treat any type of biological tissue not inconsistent with the objectives of the present disclosure. Treatment can be for aesthetic, medical, or other purposes. For example, in some cases, the biological tissue comprises skin tissue, adipose tissue, connective tissue, muscle tissue, nerve tissue, cartilage, and/or gland tissue. Further, in some embodiments, a method described herein is used to target fat cells and/or other causes of cellulite. In other cases, a method described herein is used to target tissue components causing skin laxity. In some embodiments, a method described herein is used to target lentigo or “port wine stain” tissues; tissue components causing rhytids or wrinkles; tissue containing inks from tattoos for tattoo removal purposes; glands or other tissue causing hyperhidrosis; tissue causing incontinence; glands or other tissue causing sialorrhea; scar tissue; and/or seborrheic keratosis. In some embodiments, a method described herein provides an aesthetic effect, a medical or therapeutic effect, or a combination of the foregoing. In some instances, a method described herein provides an aesthetic effect but not a medical or therapeutic effect. As understood by one of ordinary skill in the art, an aesthetic effect is an effect related primarily to a patient's appearance, with no benefit or minimal benefit to the physical health of the patient, or without treating a disease or ailment of the patient. A medical or therapeutic effect, in contrast, is an effect related primarily to treatment of a disease, ailment, or other condition of a patient that is not necessarily associated with the patient's appearance but is instead primarily related to physical health.

Methods described herein, in some cases, can also comprise or be characterized by certain additional steps or features (or a combination of additional steps and features) that can provide one or more advantages compared to other methods. For example, in some embodiments, the homogenate or homogenized tissue (of a first, second, and/or nth target volume) is fractionated within non-homogenized or healthy biological tissue in the region of biological tissue. As understood by one of ordinary skill in the art, a homogenate can refer to a suspension of cell fragments and cell constituents obtained when tissue is homogenized, such as occurs in histotripsy. In some embodiments of methods described herein, the ratio (by mass or volume) of homogenate (or homogenized or injured tissue) to non-homogenized (or healthy) tissue in the region of biological tissue is between 10:1 and 1:10, between 5:1 and 1:5, between 2:1 and 1:2, between 1:1 and 1:10, or between 1:2 and 1:5. That is, injuries or tissue damage produced by a method described herein correspond to mechanical injury points (MIPs) embedded within healthy, viable tissue. A 1:1 ratio would thus indicate that 50% of the total area, total volume, or total mass within a tissue space (or region of biological tissue) is injured or homogenized; similarly, a 1:2 ratio would indicate 33% of the total area, total volume, or total mass within a tissue space is injured or homogenized. In some cases, the percentage of non-homogenized (or healthy) tissue within a treated region of biological tissue is at least 20%, at least 30%, at least 40%, at least 50%, at least 60%, at least 70%, at least 80%, at least 90%, between 30% and 99%, between 30% and 90%, between 40% and 99%, between 40% and 90%, between 50% and 99%, between 50% and 90%, between 60 and 99%, between 60% and 90%, between 70% and 99%, or between 70% and 90%, based on the total mass or volume of tissue within the treated region. It is further to be understood that the foregoing ratios or percentages can be achieved through one treatment (or one ‘session’ or one treatment/medical care provider visit) or a plurality of separate, sequential treatments (or ‘sessions’ or treatment/medical care provider visits). For example, in some cases, up to ‘n’ separate, sequential treatments, sessions, or visits are used, wherein ‘n’ may be an integer ranging from 2 to 1000. Moreover, when an overall ratio or percentage described above is achieved through multiple treatments (or sessions or visits), the sequential treatments (or sessions or visits) can be separated by a specific time period or average time period, such as one day, one week, or one month. That is, in some cases, a method described herein is carried out over the course of or by means of periodic treatments (or sessions or visits) that may occur daily, weekly, monthly, or at some other frequency.

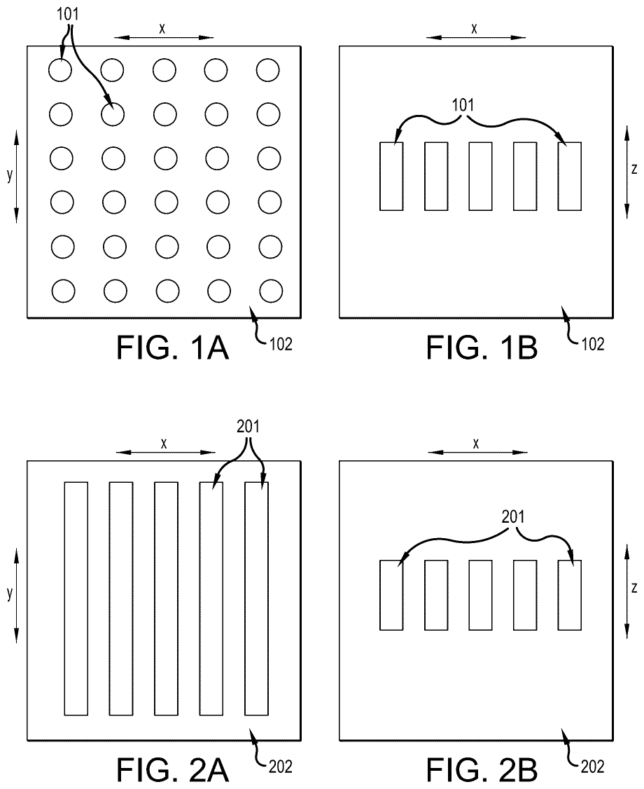

It is also possible for MIPs embedded within healthy, viable tissue to be described in terms of a “composite” structure based on connectivity between individual or distinct MIPs and/or between distinct regions of healthy tissue. For example, a fractionated tissue pattern described herein can be referred to as an “A-B” composite, where “A” refers to how many different spatial directions (e.g., as represented by the x, y and z axes) it is possible to ‘move’ along from one MIP to another MIP without ‘crossing a boundary’ of, encountering, or crossing into healthy tissue. Thus, three-dimensional ‘columns’ of MIPs in a three-dimensional ‘sea’ of healthy tissue would have an “A” value or connectivity score of 1 (movement of more than 2 mm is possible only along the height or long axis of the columns without encountering healthy tissue). Similarly, “B” refers to how many different spatial directions (e.g., as represented by the x, y and z axes) it is possible to ‘move’ along from one region of healthy tissue to another distinct region of healthy tissue without by necessity ‘crossing a boundary’ of, encountering, or crossing into a MIP. Thus, using the same example of ‘columns’ of MIPs disposed in a ‘sea’ of healthy tissue, the value of “B” or the “B” connectivity score is 3, since movement of more than 2 mm is possible in three directions (x, y, and z). FIGS. 1-4, which are further described below, illustrate various “A-B” composite patterns. In composite structures such as those illustrated in FIGS. 1-4 or other patterns of MIPs, the average distance (center-to-center or edge to edge) between individual regions of non-homogenized or healthy tissue can be no greater than 5 mm in some embodiments, such as 0.2-5 mm, in at least one dimension. In some cases, the average distance between regions of non-homogenized or healthy tissue is no greater than 5 mm in two or all three dimensions.

It is therefore to be clearly understood that homogenate or homogenized tissue that is “fractionated” within non-homogenized or healthy tissue corresponds to the result of altering or treating only a fraction of the overall tissue space (or region of biological tissue). In this context, it should be carefully noted that “fractionated” does not mean “homogenized.” In some literature related to histotripsy, the verb “to fractionate” tissue is used to mean “to homogenize” tissue. This use of “fractionate” is to be distinguished from the use of “fractionated” in the present application.

It is further to be understood that the treated region of biological tissue generally does not refer to the entire treated organism (e.g., to the entire human patient or to the human patient's body in its entirety), but instead to a specific treated area of the organism, such as an area or volume of the skin, muscle, fat, nerves, or glands of the patient having an area or volume of no greater than about 1000 cm² or 1000 cm³.

Further, in some embodiments, methods described herein do not mechanically destroy large tissue regions (e.g., a tumor), such as tissue regions having a size in at least one dimension (e.g., the x, y, or z dimension) equal to or greater than 10 mm. In some cases, methods described herein do not mechanically destroy or homogenize regions of tissue having a size in one, two, or three dimensions that is greater than 10 mm. That is, methods described herein do not necessarily mechanically destroy or homogenize all tissue within a relatively large volume, such as a volume having a size in one, two, or three dimensions of 10 mm or greater.

Methods described herein, in some cases, can produce injuries (or homogenized regions) that are localized, such that healthy tissue may grow into the space(s) created by the injuries (homogenization) produced by the methods. For example, in some instances, the homogenate or homogenized tissue areas or regions (or injuries) have a size in one, two, or three dimensions that is less than 5 mm, less than 3 mm, less than 2 mm, or less than 1 mm. It is to be understood, however, that a method described herein can produce injuries (or homogenized regions) that each have a small size individually (as indicated above), but that together have a total or combined size that is greater than the specific size limits recited in this paragraph. That is, multiple injuries (or homogenized regions) can be formed by a method described herein in a single treatment session, and these multiple or plurality of injuries (or homogenized regions) can be produced in spatial locations relative to one another that provide for coherent overall treatment of a region of tissue, while no single one of the injuries (or homogenized regions) has a size that is larger than the maximum sizes recited in this paragraph.

In addition, a target volume of a method described herein can be positioned at any desired location within the organism or human patient. For instance, a target volume can have an average or mean depth below the skin or exterior surface of the organism or human patient. In some cases, a target volume described herein has a mean or average depth of 0.1-20 mm, 0.1-15 mm, 0.1-10 mm, 0.1-5 mm, 0.1-3 mm, 0.1-2 mm, 0.2-20 mm, 0.2-15 mm, 0.2-10 mm, 0.2-5 mm, 0.2-3 mm, 0.2-2 mm, 0.5-20 mm, 0.5-15 mm, 0.5-10 mm, 0.5-5 mm, 0.5-3 mm, 0.5-2 mm, 0.7-20 mm, 0.7-15 mm, 0.7-10 mm, 0.7-5 mm, 0.7-3 mm, 0.7-2 mm, 1-20 mm, 1-15 mm, 1-10 mm, 1-5 mm, 1-3 mm, or 1-2 mm below the skin or exterior surface of the organism. Moreover, it is also possible, in some implementations, to vary the depth or location of a target volume (or a series of n target volumes, as described further herein) during the course of carrying out a method described herein.

Further, in some cases, the type and/or density of injuries (or homogenized regions) produced by a method described herein can be selected based on one or more clinically relevant factors, such as one of the following, more than one of the following, or all of the following factors: location of target volume (e.g., depth), tissue type, subject/patient demographics (e.g., age, skin type), and the clinical end goal (e.g., wrinkle reduction, skin tightening, body contouring).

FIGS. 1-4 illustrate some non-limiting examples of patterns or characteristics of injuries (or homogenized regions) that may be provided in accordance with a method described herein. Although the injuries shown in FIGS. 1-4 appear to have a repeating pattern, this is not a requirement, as the treatment may have different periodicity in the x, y, and z dimensions (or two of these dimensions) or be aperiodic in one, two, or all three dimensions.

With reference to the figures, FIG. 1 illustrates a pattern of MIPs or homogenized regions 101 produced by a method described herein according to one embodiment of the present disclosure. More specifically, FIG. 1A illustrates a pattern of MIPs or homogenized regions 101 in the coronal plane (e.g., xy-plane). FIG. 1B illustrates the same pattern of MIPs or homogenized regions 101 in the cross-sectional plane (e.g., xz-plane). The pattern of MIPs or homogenized regions 101 illustrated in FIG. 1 corresponds to a 1-3 composite fractionated region of biological tissue. In the embodiment of FIG. 1, the MIPs or homogenized regions 101 define an array of cylindrical MIPs or homogenized regions. The MIPs or homogenized regions 101 are surrounded by or embedded within a “sea” of healthy tissue 102.

Similarly, FIG. 2 illustrates a pattern of MIPs or homogenized regions 201 produced by a method described herein according to one embodiment of the present disclosure. Specifically, FIG. 2A illustrates a pattern of MIPs or homogenized regions 201 in the coronal plane (e.g., xy-plane). FIG. 2B illustrates the same pattern of MIPs or homogenized regions 201 in the cross-sectional plane (e.g., xz-plane). The pattern of MIPs or homogenized regions 201 illustrated in FIG. 2 corresponds to a 2-2 composite fractionated region of biological tissue. In the embodiment of FIG. 2, the MIPs or homogenized regions 201 define an array of rectangular, columnar MIPs or homogenized regions. The MIPs or homogenized regions 201 are surrounded by or embedded within healthy tissue 202.

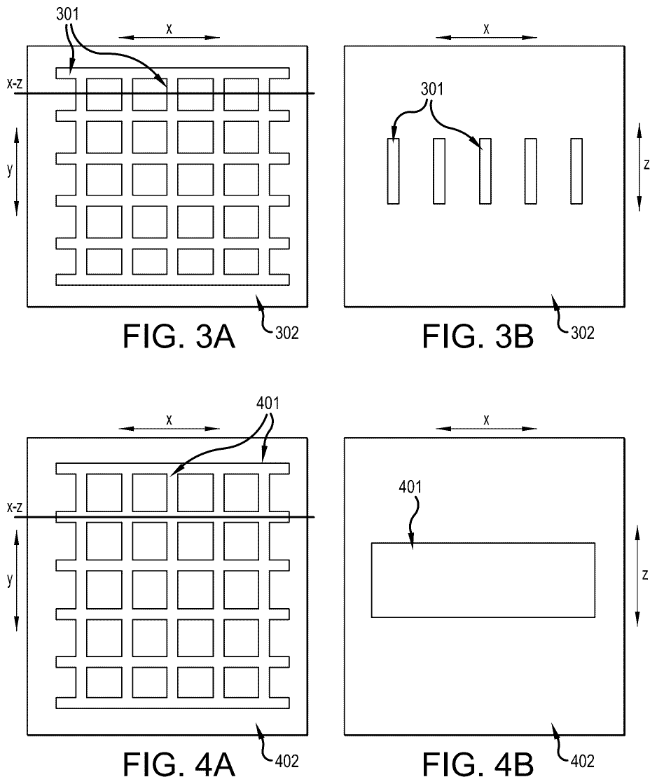

FIG. 3 illustrates a pattern of MIPs or homogenized regions 301 produced by a method described herein according to one embodiment of the present disclosure. More particularly, FIG. 3A illustrates a pattern of MIPs or homogenized regions (301) in the coronal plane (e.g., xy-plane). FIG. 3B illustrates the same pattern of MIPs or homogenized regions 301 in the cross-sectional plane (e.g., xz-plane), with the cross-section taken along the indicated x-z line in FIG. 3A. The pattern of MIPs or homogenized regions 301 illustrated in FIG. 3 corresponds to a 3-1 composite fractionated region of biological tissue. In the embodiment of FIG. 3, the MIPs or homogenized regions 301 define a periodic grid of MIPs or homogenized regions. The MIPs or homogenized regions 301 are surrounded by or embedded within healthy tissue 302.

FIG. 4 illustrates a pattern of MIPs or homogenized regions 401 produced by a method described herein according to one embodiment of the present disclosure. More particularly, FIG. 4A illustrates a pattern of MIPs or homogenized regions 401 in the coronal plane (e.g., xy-plane). FIG. 4B illustrates the same pattern of MIPs or homogenized regions 401 in the cross-sectional plane (e.g., xz-plane), with the cross-section taken along the indicated x-z line in FIG. 4A. The pattern of MIPs or homogenized regions 401 illustrated in FIG. 4 corresponds to a 3-1 composite fractionated region of biological tissue. In the embodiment of FIG. 4, the MIPs or homogenized regions 401 define a periodic grid of MIPs or homogenized regions. The MIPs or homogenized regions 401 are surrounded by or embedded within healthy tissue 402.

Moreover, in some embodiments, the ultrasound pressure wave used in a method described herein is modified after initially forming the cavitation bubble cloud. For example, in some cases, the cavitation or cavitation bubble cloud is initiated or started using a relatively high amplitude ultrasound wave or pulse and is then maintained with a lower amplitude ultrasound wave or pulse. Thus, in some implementations, a method described herein comprises reducing an amplitude or power of the ultrasound pressure wave after forming the cavitation bubble cloud, while still maintaining the cavitation bubble cloud. The maintained cavitation bubble cloud, in some cases, may be denoted as a “reduced amplitude” cavitation bubble cloud. In this manner (using a reduced amplitude cavitation bubble cloud), a method described herein can be more efficient and can generate less heating or thermal energy within the biological environment. A reduced amplitude cavitation bubble cloud can also provide better safety performance and lower likelihood of heating tissue pre-focally and/or post-focally. Further, in some cases, the reduced amplitude bubble cloud may be maintained in the same spatial location after initial formation of the bubble cloud, or the bubble cloud may subsequently be moved to another location through either mechanical or electronic steering and focusing of the pressure wave at the lower amplitude. In some such cases in which the focus of the reduced amplitude bubble cloud is moved subsequent to formation, the rate of movement is sufficiently slow to maintain the bubble cloud at the reduced amplitude. In some embodiments, the target or permissible rate of movement may be related to the size of the bubble cloud (including in the direction of movement), the intensity distribution within the ultrasound beam, the pulse repetition frequency (PRF), and/or the time required to homogenize tissue. Without being bound by theory, it appears that in some embodiments the reduced amplitude bubble cloud creates pressure fields that give rise to low pressure zones throughout the area being treated. Bubble cloud trailing could be achieved, for example, by using the low pressure gradient to effect movement of the bubble cloud as the ultrasound beam is moved from one spatial location to another such as, for example, by moving the bubble cloud in the direction of transducer movement. In some embodiments, the amplitude, prf, and/or other conditions or variables can be manipulated or varied until bubbles are detected. In some embodiments, the bubble cloud may be moved spatially at a rate that facilitates maintenance of the cloud.

In some embodiments of a method described herein, a pressure wave may be positioned in n target volumes of n regions of biological tissue to form n cavitation bubble clouds in the n target volumes. The n cavitation bubble clouds can, in some embodiments, be maintained for a predetermined or desired time period, while the transducer is moved (i.e., translated or rotated). The transducer can be moved manually, mechanically, electronically, or by any other means available. In some embodiments, the transducer is sufficiently moved (in other words, sufficiently rotated or translated) to produce homogenate or to produce homogenized tissue while maintaining the bubble cloud that tracks the movement of the transducer. As a result, it is possible to also produce homogenate or homogenized tissue within another target volume of the n target volumes. In such embodiments, the bubble cloud can be “dragged” through tissue to create homogenate or homogenized tissue in multiple target volumes and/or multiple regions of biological tissue along the path of the bubble cloud.

In addition, in some embodiments of a method described herein, a reduced amplitude cavitation bubble cloud can be used throughout a majority of the temporal duration of a method described herein (e.g., throughout a majority of the total treatment time). For instance, in some cases, a reduced amplitude bubble cloud can be used during at least 80%, at least 70%, or at least 60% of a total time of carrying out a method or treatment (or applying ultrasound) described herein. In some instances, a reduced amplitude bubble cloud is used during 10-99%, 10-90%, 10-80%, 10-70%, 20-99%, 20-90%, 20-85%, 20-80%, 20-70%, 20-60%, 20-50%, 30-99%, 30-90%, 30-85%, 23-80%, 30-70%, 30-60%, 30-50%, 40-99%, 40-90%, 40-85%, 40-80%, 40-70%, 40-60%, 50-99%, 50-90%, 50-85%, 50-80%, 50-70%, 60-99%, 60-90%, 60-85%, 60-80%, or 60-70% of a total treatment time or a total time of carrying out a method (or applying ultrasound) described herein.

Additionally, in some embodiments, the pressure wave used in a method described herein is a composite pressure wave comprising a high frequency component wave (or waveform) and a low frequency component wave (or waveform). The high and low frequency waves may have the same or different functions, including imaging, detection, and/or therapy. For instance, in some embodiments, a high frequency component wave may be used for imaging and/or detection, and the low frequency component wave may be used for therapy. In other embodiments, the high frequency component wave may be used for therapy, whereas the low frequency component wave may be used for imaging and/or detection. In yet other embodiments, both frequencies may be employed for therapy. Any combination of functions for the high and low frequency waves not inconsistent with the technical objectives of the present disclosure may be employed without departing from the scope of the invention.

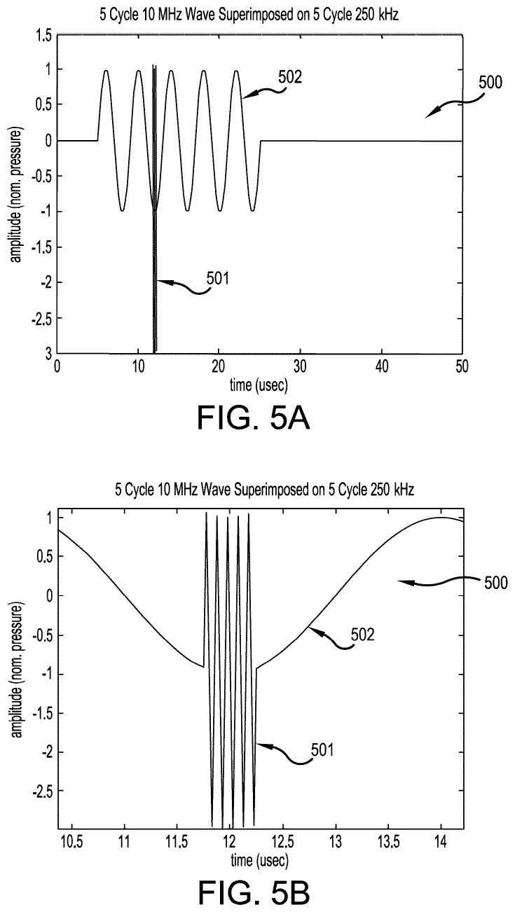

FIG. 5A illustrates such a composite pressure wave. With reference to FIG. 5A, a composite pressure wave 500 comprises a high frequency component wave 501 and a low frequency component wave 502, where it is understood that the terms “high” and “low” are relative to one another (that is, the “high” frequency wave has a higher frequency than the “low” frequency wave). FIG. 5B illustrates an enlarged portion of FIG. 5A. As illustrated in FIG. 5A, a 5-cycle 10 MHz wave (the high frequency component wave, 501) is superimposed on a 5-cycle 250 KHz wave (the low frequency component wave, 502). Other combinations of high and low frequency component waves are also possible. Specifically, although FIGS. 5A and 5B show a low frequency wave longer in time than the high frequency wave, other combinations are possible without departing from the scope of this disclosure. For instance, in some embodiments, the high frequency wave may be longer than the low frequency wave.

As noted above, in some embodiments, high frequency waves and low frequency waves may serve multiple purposes. In some embodiments, the high frequency wave may be used to sustain the cloud bubble, to detect Doppler shifts from movement, and/or to detect oscillations of the bubbles through scattering. Any combination of therapy, imaging and/or detection actions may be employed without departing from the fundamental spirit and scope of the invention.

In some embodiments, the low frequency wave may be 1/50, 1/40, 1/30, 1/20, 1/10, ⅕, ½, and/or any value or ratio in between these values, of the high frequency wave in terms of frequency thereof. These ratios represent the ratio of the two frequencies, wherein the first number is the low frequency and the second number is the high frequency. For example, comparing a 500 kHz low frequency wave with a 10 MHz high frequency wave gives a ratio of 1/20 (500 kHz/10,000 kHz). The bigger the ratio, the easier it is to fit the high frequency wave in the trough or peak of the low frequency wave. In some embodiments, a peak amplitude of the low frequency wave may be 1/50, 1/40, 1/30, 1/20, 1/10, ⅕, ½, or 0.99 of a required cavitation threshold. The cavitation threshold is tissue-dependent. Cavitation thresholds for various tissues are disclosed in Tables 1 and 4 herein.

In some embodiments, a low frequency wave may originate from the same transducer as the high frequency wave, whereas in other embodiments, the low frequency wave may originate from a different transducer than the high frequency wave. In some embodiments, the determination regarding transducer use for originating the low frequency wave is based on a target timing of the waveforms. When the target timing of the waveforms is satisfied, the high frequency wave can be placed at a desired or target position on the low frequency wave.

Additionally, in the embodiment of FIG. 5A, the high frequency component wave is ‘riding’ the trough of the low frequency component wave. That is, the high frequency component wave is disposed or positioned within a trough of the low frequency component wave, such that, in some embodiments, all cycles of the high frequency component wave ‘fit within’ the trough of the low frequency component wave. In other embodiments, one or more cycles of the high frequency wave, but not all cycles, is or are in the trough of at least one cycle of the low frequency wave.

In terms of placement of the high frequency wave and the low frequency wave, in some embodiments, the high frequency wave component may be disposed or positioned within a peak of the low frequency component wave. In some embodiments, positioning the high frequency wave component within a peak of the low frequency component wave results in all cycles of the high frequency component wave to “fit within” the peak of the low frequency component wave. As such, in some embodiments, the high frequency component wave may be “riding” on the peak of the low frequency component. Moreover, in some embodiments, such “riding” may result in enhanced support of the bubble cloud and/or enhanced non-linear heating, especially in instances in which bubbles are already at the focus.

Moreover, in some implementations, cycles of the high frequency component wave can be spaced apart from one another such that at least one cycle of the high frequency component wave is disposed within a trough (or a plurality of troughs) of at least one cycle of the low frequency component wave. In some embodiments, one or more cycles of the high frequency component wave is/are positioned in a trough of one or more cycles of the low frequency component wave. In some embodiments, the high frequency component wave is positioned in a trough of the low frequency component wave.

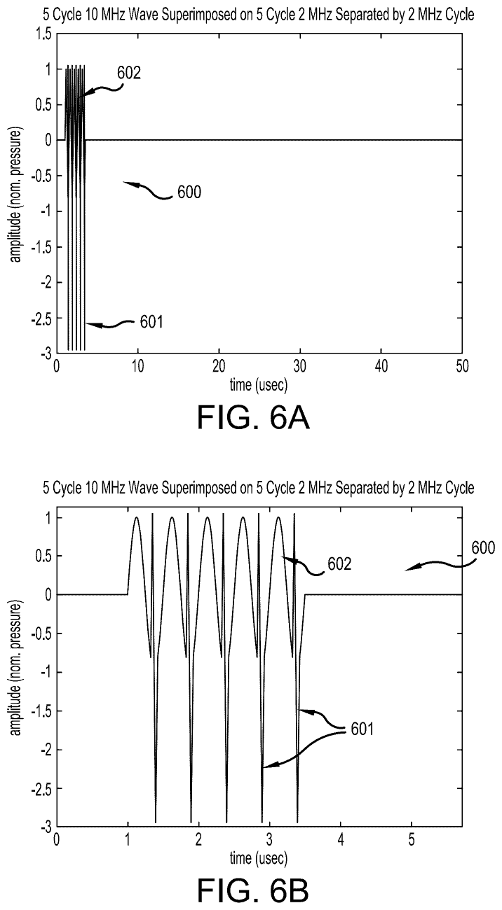

For example, as illustrated in FIG. 6A, one cycle of the high frequency component wave can be disposed within each trough of the low frequency component wave. In some embodiments, this enables the lower frequency wave to achieve a higher frequency. Further, in some such embodiments, cycles of the high frequency component wave are separated by one or more ‘waiting’ cycles. In some embodiments, the waiting time may promote greater growth and/or contraction of cavitation bubbles, which may lead to a more efficient treatment and/or a safer treatment.

With reference to FIG. 6A, a composite pressure wave 600 comprises a plurality of cycles of a high frequency component wave 601 and a plurality of cycles of a low frequency component wave 602. FIG. 6B illustrates an enlarged portion of FIG. 6A. As illustrated in FIG. 6A, a 5-cycle 10 MHz wave (the high frequency component wave, 601) is superimposed on a 5-cycle 2 MHz wave (the low frequency component wave, 602). Specifically, one cycle of the high frequency component wave 601 is disposed in each of the illustrated troughs of the low frequency component wave 602, and the high frequency cycles 601 are separated by a time period equivalent to a 2 MHz cycle (that is, adjacent cycles of the high frequency component wave are separated by the frequency of the low frequency component wave, which is 2 MHz in this example). Component pressure waves such as that illustrated in FIG. 6A can permit multiple troughs of the low frequency component wave to each contain one or more high frequency component waves ‘riding’ on the low frequency component wave. FIG. 6B illustrates an enlarged portion of FIG. 6A.

In some embodiments of methods described herein, it is also possible for a high frequency component wave or waveform to be disposed in a compressional part of a low frequency component wave or waveform of a composite wave, where it is to be understood that the compressional part of a wave or waveform is generally the part of the wave or waveform where the conversion of energy at the fundamental frequency is converted to higher frequency harmonics. Providing such a composite wave such as described herein can, in some implementations, provide one or more advantages. For example, in some such instances, the probability of creating a mechanical bubble, sustaining a bubble cloud, and/or creating more bubbles is lowered and the probability of creating non-linear harmonics with increased heating near the intended treatment location is increased. Thus, in some embodiments described herein, generating additional heat at the focus is possible, since absorption increases with frequency and most of the harmonics are produced near the ultrasound focus. Additionally, once a threshold has been reached for creation of a bubble cloud as described herein, other advantages may be obtained by placing a high frequency component wave in the compressional part of the low frequency wave. For example, and not intending to be bound by theory, pulse inversion can occur from reflection off the existing bubbles of a formed bubble cloud, which can help maintain the bubble cloud at a reduced intensity. Promoting more nonlinear heating around the bubble cloud is also possible in some cases.

In general, the high frequency component wave and the low frequency component wave can each have any frequency not inconsistent with the technical objectives of the present disclosure. In some embodiments, for example, the high frequency component wave and the low frequency component wave have frequencies in accordance with Equation 1:

$$p = \frac{{A}_{High} - {A}_{Low} sin ( \frac{3 π}{2} + \frac{( 2 n - 1 ) {f}_{Low} π}{2 {f}_{High}} )}{{A}_{High} + {A}_{Low}} , \qquad ( Equation 1 )$$

where p is a ratio of the magnitude of the last negative peak pressure (i.e., the negative peak pressure for the last cycle of a wave) relative to the maximum negative pressure (i.e., the sum of A_{High} and A_{Low}), n is the number of cycles of the high frequency component wave in one pulse of the ultrasound pressure wave, f_{low} is the frequency of the low frequency component wave, f_{high} is the frequency of the high frequency component wave, A_{Low} is the amplitude of the low frequency excitation, and A_{High} is the amplitude of the high frequency excitation.

Equation 1 above is based, in part, in the relationship or ratio between the least negative pressure in a burst relative to the greatest negative pressure in that burst. In some embodiments, it is possible to determine whether a threshold pressure has been exceeded throughout the entire burst using Equation 1, since the ability to cavitate can be dependent on negative pressures (and, as a result, is dependent on efficacy).

Additionally, in some cases, f_{high} is a pre-selected treatment frequency, where a “treatment” frequency for references purposes herein is a frequency selected for its therapeutic or clinical effects. A treatment frequency may also be selected for its performance attributes such as penetration depth, beam width, and the like. For example, in some embodiments, f_{high} is between 1 MHz and 15 MHz, such as 5 MHz. Other values of f_{high} are also possible. Further, in some instance, f_{low} is between 0.1 and 5 MHz, between 0.1 and 3 MHz, less than 5 MHz, less than 3 MHz, or less than 2 MHz. Other values of f_{low} are also possible, as understood by one of ordinary skill in the art.

Moreover, in some embodiments, the frequency of the high frequency component wave (the “high frequency”) and the frequency of the low frequency component wave (the “low frequency”) can be selected based on one or more of the following: a desired or pre-selected number of cycles of the high frequency component wave per trough of the low frequency component wave; a desired, pre-selected, or allowable variance, wherein said “variance” is defined as the ratio of the last negative pressure to the greatest maximum pressure in a burst; and a desired or pre-selected treatment frequency. Table 2 below illustrates some non-limiting examples of possible low frequency values, based on a 5 MHz high frequency (treatment frequency) for different numbers of cycles in the trough and different selections of permissible signal variance.

TABLE 2
Low Frequency Values (in kHz) for a 5 MHz Treatment Frequency
for Different # of Cycles at the Trough and Permissible
Variance (Variance = Ratio of Negative Pressures in a Burst)

| | |
|---|---|
| # of | Allowable Variance |

| Cycles | 0.05 | 0.1 | 0.2 | 0.5 |
|---|---|---|---|---|
| 1 | 1440 | 2050 | 2950 | 5000 |
| 2 | 480 | 682 | 983 | 1670 |
| 5 | 159 | 227 | 328 | 555 |
| 10 | 75.5 | 107 | 155 | 263 |

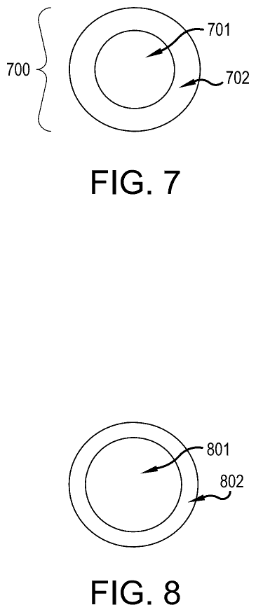

Composite pressure waves such as described above can be provided in any manner not inconsistent with the technical objectives of the present disclosure. In some cases, a composite pressure wave is provided or formed by a hybrid ultrasound transducer or array of transducers, such as a hybrid ultrasound transducer or array comprising a low frequency transducer component and a high frequency transducer component. In some instances, a high frequency transducer component is positioned around or surrounds a low frequency transducer component. One such arrangement is illustrated in FIG. 7, which illustrates a top view of a hybrid ultrasound transducer or array in accordance with one embodiment described herein. With reference to FIG. 7, a hybrid ultrasound transducer or array 700 comprises a high frequency transducer component 701 surrounding a low frequency transducer component 702. Other structures or arrangements are also possible. Specifically, the outer transducer could be the low frequency or high frequency transducer, and the inner transducer would be the opposite. The arrangement of the embodiment of FIG. 7 may be especially advantageous for providing mechanical injuries having a narrow effective width. Moreover, although FIG. 7 shows that the high frequency and low frequency transducers are co-located in the same region, in another embodiment, one or more high locations from the intended target tissue such that acoustic energy from each transducer can arrive coherently at the intended target tissue. For example, the high frequency transducer(s) can be located orthogonal to the low frequency transducer(s) or otherwise spaced apart from the low frequency transducer(s), as opposed to being physically overlapping in at least one dimension. It is also possible to use a transducer that may be excited with both the low frequency and the high frequency components simultaneously, based on the bandwidth and resonance behavior of the transducer. For example, as understood by one of ordinary skill in the art, a broadband transducer or dual frequency transducer (e.g., a bilayer transducer) may be designed, built, and used in a method described herein.

It is further to be noted that methods described herein can provide various levels of heating to a region of biological tissue. Controlling the thermal effect of an ultrasound-based method can provide advantages compared to other methods. In some cases, a method described herein provides a relatively low amount of thermal energy. For example, in some embodiments, maintaining a cavitation bubble cloud in a target volume (or plurality or series of target volumes) does not deliver a thermal dose to tissue within the target volume (or plurality or series of target volumes) that is sufficient to create coagulation, denaturation, apoptosis, or any other type of cell death. It is to be understood that denaturation refers to a permanent change of a protein's structure, while coagulation refers to the process of converting liquid molecules into a solid or semi-solid state. Denaturation is the first step in coagulation. Coagulation may be more visible, but less reversible, than denaturation. Apoptosis, on the other hand, refers to a process of programmed cell death, and may be used to rid the body of cells that have been damaged beyond repair.

It will be understood that a thermal dose can comprise a total amount of heat or thermal energy (or acoustic energy converted to heat through absorption) delivered to or experienced by tissue that is sufficient to provide a given tissue response, such as coagulation, denaturation, or apoptosis, or a given probability of tissue death (e.g., 60% or higher). Moreover, the thermal dose can be determined in accordance with a bioheat function or other hyperthermia parameter. For example, in some instances, a thermal dose is based on the Arrhenius equation. Further, in some embodiments, a thermal dose described herein corresponds to a total equivalent minutes at 43 degrees C. of greater than 240 minutes, as described in Dewhirst et al., “Thermal Dose Requirement for Tissue Effect: Experimental and Clinical Findings,” Proc SPIE Int Soc Opt Eng. 2003 Jun. 2; 4954:37-. doi: 10.1117/12.476637. Avoiding delivery of a thermal dose as described herein, in some cases, can provide a safer, more effective treatment, particularly for facilitating the survival and growth of healthy tissue.

In some embodiments, to limit or avoid a thermal effect or delivery of a thermal dose, a method described herein uses an intentionally limited duty cycle of an ultrasound pulse. For example, in some cases, the pulse duty cycle is less than 10%, less than 5%, less than 2%, or less than 1%.

As an alternative, if desired, it is also possible for a method described herein to deliver a thermal dose that is sufficient to create coagulation, denaturation, or apoptosis. Thus, in some cases, maintaining a cavitation bubble cloud in a target volume (or plurality or series of target volumes) delivers a thermal dose to tissue within the target volume (or plurality or series of target volumes) that is sufficient to create coagulation, denaturation, or apoptosis. Additionally, in some such instances, the thermal dose is sufficient to thermally ablate all or substantially all (e.g., at least 90%, at least 95%, at least 98%, or at least 99% of) tissue within the target volume (or plurality or series of target volumes). Further, in some cases, a thermal dose is delivered before a mechanical injury (or homogenized region) is created by the bubble cloud. In such instances, in some embodiments, delivering a thermal dose and raising the temperature within the target volume can reduce the cavitation threshold of the target volume and permit the use of a lower peak negative pressure. In other instances, a thermal dose is delivered after a mechanical injury (or homogenized region) is created by the bubble cloud. In some such embodiments, delivery of a thermal dose after the creation of a mechanical injury can expedite coagulation within the mechanical injury. In still other implementations, a method described herein delivers a thermal dose and creates a mechanical injury simultaneously. In some such cases, not intending to be bound by theory, it is believed that the cavitation cloud locally enhances ultrasound energy absorption.

The thermal dose may be given or delivered before, during, or after homogenization of healthy tissue. In addition, in some cases, a method described herein further comprises forming a thermal coagulation zone around a target volume, which in some embodiments can contain homogenized tissue or homogenate. In some embodiments, tissue denaturation, tissue coagulation, and/or apoptosis may increase the acoustic attenuation of tissue. In some embodiments, thermally coagulating or denaturing a target volume in an area immediately below a treatment area may enhance cavitation. Without being bound by theory, it appears that in some embodiments, when denatured or coagulated tissue is just below a target treatment area (i.e., just below an area where an intended procedure, such as histotripsy, is performed), reflection may occur off of such denatured or coagulated tissue, thus improving cavitation. Increased acoustic attenuation may also be used, in some embodiments, to prevent propagation of acoustic energy to deeper tissue areas.

In still other embodiments, it is possible to use precise delivery of thermal energy, including in combination with cavitation, to create more complex structures within a region of biological tissue. For example, in some cases, a method described herein further comprises forming a thermal coagulation zone (or a plurality or series of thermal coagulation zones) around a target volume (or a plurality or series of target volumes) described herein. Such a thermal coagulation zone can surround or at least partially encapsulate homogenized tissue within a target volume. In some instances, a combination of mechanical cavitation with surrounding thermal coagulation is provided using the same or different pressure waves, or using a composite pressure wave, or using a sequence of target volumes. In some embodiments, for example, a first target volume described herein is homogenized without delivery of a thermal dose, and a second target volume is thermally coagulated by delivery of a thermal dose.

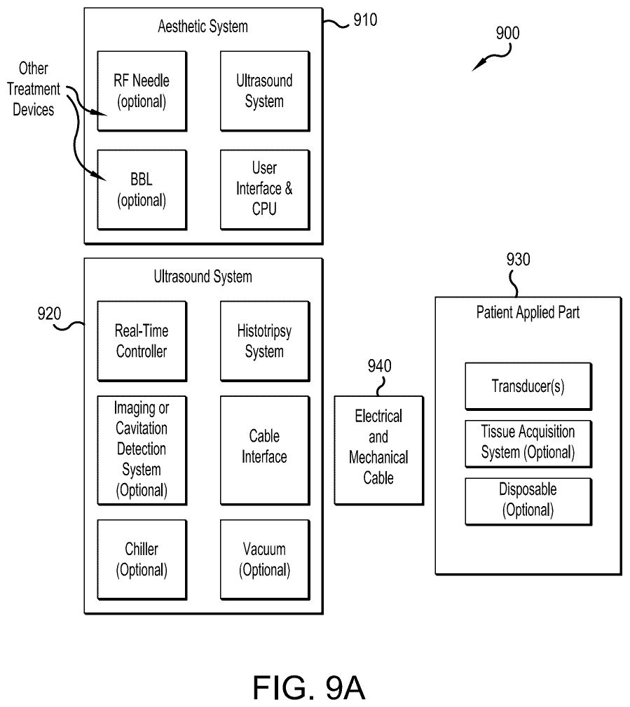

Moreover, in such cases, the second target volume can surround or encapsulate the first target volume, as illustrated in FIG. 8, which shows a top view of a first target volume and a second target volume treated in accordance with one embodiment of methods described herein. With reference to FIG. 8, the first target volume 801 has a circular cross-section when viewed from the top (e.g., along a line orthogonal to the exterior surface or skin of the patient) and is generally cylindrical in shape. However, other shapes are also possible. The first target volume 801 comprises homogenate that has not been coagulated. The second target volume 802 surrounds the first target volume 801 concentrically, such that the first target volume 801 and the second target volume 802 form concentric cylinders within the region of biological tissue. The second target volume 802 comprises coagulated tissue, such as obtained when a method described herein is carried out with delivery of a thermal dose to the second target volume 802, where the thermal dose is sufficient to coagulate the tissue within the second target volume 802. In some embodiments, thermal denaturation or coagulation may be limited to the surface perimeter around the bubble cloud. In some embodiments, thermal denaturation or coagulation may occur within the treated volume. In some embodiments, thermal denaturation or coagulation may occur in both the surface perimeter around the bubble cloud and within the treated volume.

Moreover, in some embodiments, a method described herein can be carried out in combination, conjunction, series, or parallel with one or more other methods for treating a patient. For example, in some cases, an ultrasound-based method of treatment described herein can be used in combination with a light-based treatment such as a laser-based or broadband light (BBL)-based treatment, or a microneedle-based treatment. In some instances, an ultrasound-based method of treatment described herein can be used in combination with one or more of the methods and systems described in U.S. Patent Application Publication 2001/016732; U.S. Patent Application Publication 2019/125445; U.S. Patent Application Publication 2021/282855; U.S. Pat. No. 6,575,964; 6,770,069; 11,071,588; 11,213,350; or 11,219,485, the contents of each of which are hereby incorporated by reference in their entireties.

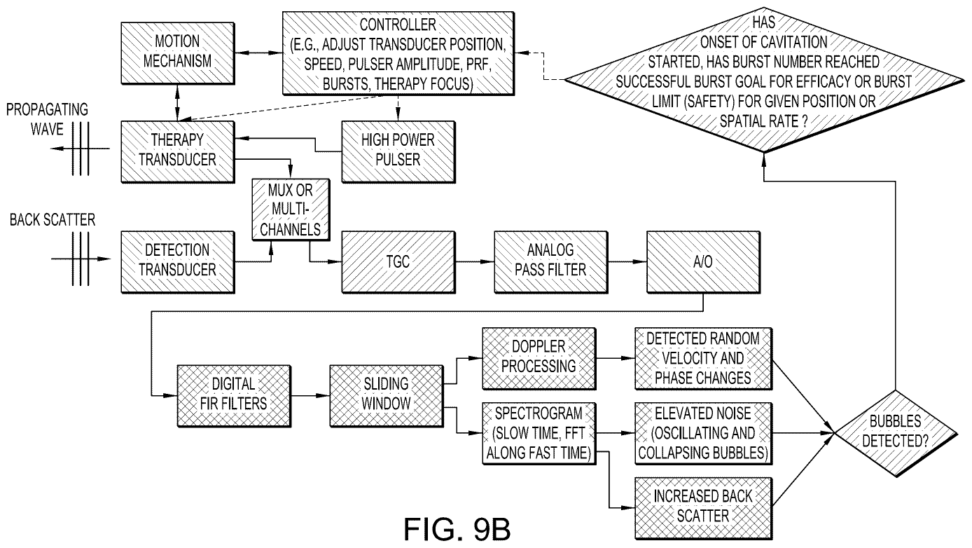

Turning again to the drawings, it is to be understood that steps of methods according to the present disclosure can be carried out in any manner and using any systems, equipment, hardware, and/or software not inconsistent with the technical objectives of the present disclosure. For example, with reference to FIG. 9A, this figure depicts aspects of a treatment system 900 in accordance with various embodiments of the present disclosure. Treatment system 900 can include a plurality of devices, components, engines, and/or modules. For example, a treatment system 900 can comprise an ‘aesthetic’ component, system, or subsystem 910. A treatment system 900 can also comprise an ‘ultrasound’ component, system, or subsystem 920, and a ‘patient applied’ part, component, system, or subsystem 930. Additionally, a system 900 can further include one or more connector parts, components, systems, or subsystems 940.

It is to be understood that the ‘aesthetic’ system 910 can provide aesthetic treatment to the patient, in addition to or in combination with ultrasound treatment provided by the overall system 900. For example, in some cases, the aesthetic system 910 can provide wrinkle removal, skin tightening, body contouring, and/or other treatments, separate and apart from histotripsy provided by the overall system 900. Thus, in some embodiments, the aesthetic system 910 comprises devices or components such as radiofrequency needle or microneedle components, laser or broadband light (BBL) components, an ultrasound component that is distinct from the ultrasound system providing histotripsy, and/or one or more controllers or user interfaces. More particularly, in some cases, an aesthetic system 910 may include a device that delivers electromagnetic radiation (e.g., a laser beam or BBL beam) to biological tissue for ablation, non-ablation, or other therapeutic purposes, or a device that provides chemical, electrical, mechanical, or electromechanical treatment to biological tissue, such as using microneedles. In some embodiments, a system 900 may be a “hybrid” system, comprising multiple features, such as one or more microneedle(s), detection transducer(s), therapy transducer(s), additional ultrasound components, such as additional transducer(s), tissue acquisition system(s), control device(s), source(s) of radiation, such as sources of electromagnetic radiation, and/or broadband light therapy system(s).

The ‘ultrasound’ system 920 of the overall system 900 can provide ultrasound for histotripsy, as described in the present disclosure. Thus, the ‘ultrasound’ system 920 can include, amongst other components, one or more ultrasound transducers or other source(s) of ultrasound for providing one or more ultrasound beams/pressure waves and one or more display devices, such as a monitor for displaying ultrasound imaging results in real time. The ‘ultrasound’ system 920 can also include one or more imaging or cavitation detection devices or sub-systems, which may be an optical imaging device or system, such as a spectrophotometer or visible light (e.g., RGB) camera, a thermal or infrared (IR) camera, an optical coherence tomography (OCT) device or system, a multi-photon imaging device or system, a reflectance confocal microscopy (RCM) device or system, a wide band piezoelectric receiver, or any other imaging system not inconsistent with the technical objectives of this disclosure. In some embodiments, ultrasound transducers can be used as cavitation detection devices. In some embodiments, the same one or more transducer(s) used to provide ultrasound beams can also be used to detect the onset of cavitation. When the same transducer is employed to provide ultrasound beams and to detect cavitation onset, the transducer can be configured with both a “transmit” mode (to provide and transmit ultrasound beams) and a “receive” mode (to receive or detect acoustic cavitation emissions).

Additionally, an ultrasound system 920 can further comprise a control device (e.g., a real-time controller) to maintain the cavitation bubble cloud in the target volume for a time period sufficient to produce homogenate (or to homogenize tissue, or to produce homogenized tissue) within the target volume. The control device can also control and/or coordinate other components of the overall system 900, the ultrasound system 920, or another subsystem, in addition to the ultrasound transducer(s). For example, the control device might also control the display device(s), the imaging device(s), and/or the additional treatment device(s) of the ‘aesthetic’ system 910. Moreover, the ultrasound system 920 can further include a mapping component, which may include a content repository, which can be a plurality of repositories, and which can be in operable communication with a treatment device, control device, and any associated engines or modules.

An overall system 900 can also comprise a ‘patient applied’ system 930, which is in contact with or otherwise disposed on or near the patient treated by the overall system 900. For example, the ‘patient applied’ system 930 may comprise one or more ultrasound transducers (which may help carry out ultrasound/histotripsy treatment by the overall system 900 or by the ultrasound system 920), one or more tissue acquisition systems (such as a vacuum), and any disposable component. The overall system 900 can further include one more connector parts (940), such as one or more electrical and/or mechanical cables to connect various components of various systems/subsystems.

An overall system 900 can also comprise a mechanism that sends test pulses, or a ramped amplitude of test pulses, when bubbles are first formed, such that the overall system 900 detects the onset of bubble formation. It is to be understood that a test pulse is or could be a sequence of pulses of increasing amplitude. These test pulses also have significantly less ‘bursts’ so as not to create a therapeutic effect in tissue. In other words, test pulses are pulses of varying amplitude, duration, and/or PRF that are used to determine necessary cavitation amplitude, without creating a therapeutic effect in a target tissue (such that changes are reversible). In some embodiments, detection of bubble formation signals to the system that a determination must be made as to how to treat a tissue of interest. As such, in some embodiments, it is possible to manipulate system parameters such that, once bubbles are detected, the overall system 900 treats a specific target volume in a specific region of tissue in a particular way, without affecting other target volumes and/or other regions of tissue.

FIG. 9B illustrates a system according to another embodiment herein. Specifically, FIG. 9B illustrates the interaction between a histotripsy system, imaging/detection system, real-time controller, transducer, and CPU according to an embodiment herein. As shown in FIG. 9B, a therapy transducer can be connected to a motion mechanism. The motion mechanism is designed to move the transducer over a target treatment region. In some embodiments, the transducer moves in a “back and forth” fashion along a line. In other embodiments, the transducer may move in a circle, serpentine, or any other non-linear manner not inconsistent with the technical objectives of the present disclosure.

In some embodiments, a detection transducer may be used as a passive listening device or in a pulse-echo mode to “interrogate” a treated region or volume. Specifically, a high frequency pulse may “ride” on a low frequency pulse as described elsewhere in this disclosure, wherein the low frequency pulse acts as an “interrogation” pulse and, if and when bubbles are created, it becomes possible to distinguish the high frequency from the low frequency, by “listening” to the bubbles and by using backscattering as a guide.

In the embodiment of FIG. 9B, a high power pulser is connected to a therapy transducer. In some embodiments, ultrasound backscatter on the therapy transducer and/or the detection transducer may be used to guide treatment. In some embodiments, a multiplexer may be used to switch between a detection transducer and a therapy transducer. In some embodiments, it is not necessary to use a multiplexer, such as when both a detection transducer and a therapy transducer are used simultaneously, since the system would have two paths for receiving backscattered signals.

In some embodiments, backscattered signals would follow a similar path to other ultrasound imaging systems. For instance, time gain control (TCG) raises the amplitude of the signal based on a delay time and/or signal depth. The backscattered signal can be, in some embodiments, further conditioned through an analog pass filter, an anti-aliasing filter, or similar filter, and subsequently digitized.

In some embodiments, a digitized signal may be used to generate a spectral Doppler signal, and/or to detect an increase in noise floor from the bubbles. In some embodiments, a signal (i.e., a Doppler signal) may be sent to the input of an artificial neural network, where it can be “trained” to detect bubbles based on the inputs. In such cases, computations and/or implementation of the neural network may take place in a CPU (central processing unit). In some embodiments, computations and/or implementation of the neural network may take place in a FPGA (field-programmable gate array), such as, for instance, when faster speeds are required. In some embodiments, computations may be used to determine whether bubbles are present, and/or whether process parameters should be adjusted to obtain a cloud bubble and/or to sustain a cloud bubble. Non-limiting examples of system parameters that can be monitored and/or manipulated include transducer position, focal position, pulser amplitude, PRF, bursts, therapy focus, and combinations thereof.

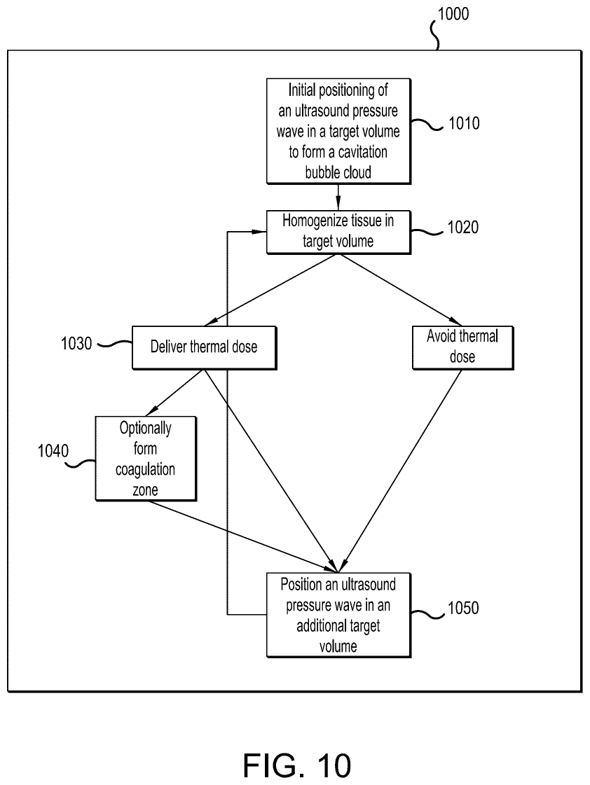

Turning now to FIG. 10, a flow diagram is provided illustrating one example method 1000 for treating a patient or region of biological tissue or a component of tissue. It is contemplated that method 1000 and other methods described herein are not limited to those illustrated, and can incorporate other blocks or steps at any point in the method in accordance with the present disclosure. At step 1010, an ultrasound pressure wave is positioned in a target volume to form a cavitation bubble cloud in the target volume. The target volume can be based on or defined by a point focus of the pressure wave or a line focus of the pressure wave. Additionally, the ultrasound pressure wave can have any other characteristics of, or be any type of, a pressure wave described herein. For instance, the pressure wave can be a composite pressure wave. At step 1020 the cavitation bubble cloud is maintained in the target volume for a time period sufficient to produce homogenate as described herein. In some cases, for instance, at step 1020 the ultrasound intensity is reduced. Optionally, at step 1030, a thermal dose can either be intentionally delivered to biological tissue in the target volume or its environment, or intentionally avoided. In the event a thermal dose is delivered, it is possible (if desired) to also form a coagulation zone or to coagulate tissue in the target volume (step 1040). Subsequently, at step 1050, an ultrasound pressure wave can be positioned in one or more additional target volumes, either in series or in parallel, or in any other manner desired to provide a particular treatment. For example, in some cases, periodic or aperiodic MIPs are produced by the method 1000.

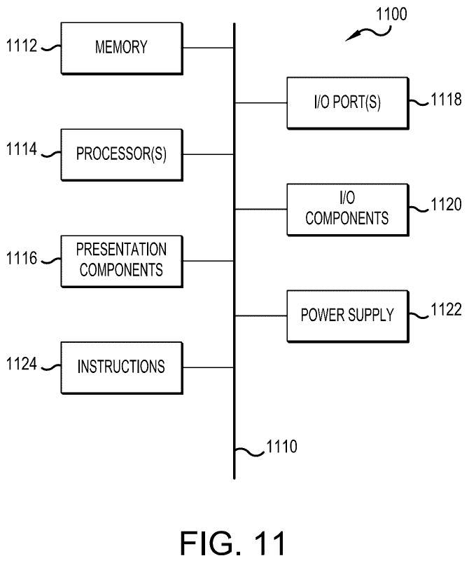

FIG. 11 provides an illustrative operating environment for implementing embodiments of the present disclosure, which is shown and designated generally as computing device 1100. Computing device 1100 is merely one example of a suitable computing environment and is not intended to suggest any limitation as to the scope of use or functionality of the invention. Neither should the computing device 1100 be interpreted as having any dependency or requirement relating to any one or combination of components illustrated.

Embodiments of the invention can be described in the general context of computer code or machine-useable instructions, including computer-executable instructions such as program modules, being executed by a computer, FPGA, or other machine (virtual or otherwise), such as a smartphone or other handheld device. Generally, program modules, or engines, including routines, programs, objects, components, data structures etc., refer to code that performs particular tasks or implements particular abstract data types. Embodiments of the invention can be practiced in a variety of system configurations, including hand-held devices, consumer electronics, general-purpose computers, more specialized computing devices, etc. Embodiments of the invention can also be practiced in distributed computing environments where tasks are performed by remote-processing devices that are linked through a communications network.

FIG. 11 shows an exemplary embodiment of a “User Interface & CPU” block within an aesthetic system (such as aesthetic system 910 in FIG. 9). With reference to FIG. 11, computing device 1100 includes a bus 1110 that directly or indirectly couples the following devices: memory 1112, one or more processors 1114, one or more presentation components 1116, input/output ports 1118, input/output components 1120, and an illustrative power supply 1122. In some embodiments, devices described herein utilize wired and rechargeable batteries and power supplies. Bus 1110 represents what can be one or more busses (such as an address bus, data bus or combination thereof). Although the various blocks of FIG. 11 are shown with clearly delineated lines for the sake of clarity, in reality, such delineations are not necessarily so clear and these lines can overlap. For example, one can consider a presentation component such as a display device to be an I/O component as well. Also, processors generally have memory in the form of cache. It is recognized that such is the nature of the art, and reiterate that the diagram of FIG. 11 is merely illustrative of an example computing device that can be used in connection with one or more embodiments of the present disclosure. Distinction is not made between such categories as “workstation,” “server,” “laptop,” “hand-held device,” etc., as all are contemplated within the scope of FIG. 11 and reference to “computing device.”

Computing device 1100 typically includes a variety of computer-readable media. Computer-readable media can be any available media that can be accessed by computing device 1100, and includes both volatile and non-volatile media, removable and non-removable media. By way of example, and not limitation, computer-readable media can comprise computer storage media and communication media.

Computer storage media include volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 1100. Computer storage media excludes signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner at to encode information in the signal. By way of example, and not limitation, communication media includes wired media such as a wired network or direct-wired connection, and wireless media such as acoustic, RF, infrared, NFC, Bluetooth and other wireless media. Combinations of any of the above should also be included within the scope of computer-readable media.

Memory 1112 includes computer storage media in the form of volatile and/or non-volatile memory. As depicted, memory 1112 includes instructions 1124 that when executed by processor(s) 1114 are configured to cause the computing device to perform any of the operations described herein, in reference to the above discussed figures, or to implement any program modules described herein. The memory can be removable, non-removable, or a combination thereof. Illustrative hardware devices include solid-state memory, hard drives, optical-disc drives, etc. Computing device 1100 includes one or more processors that read data from various entities such as memory 1112 or I/O components 1120. Presentation component(s) 1116 present data indications to a user or other device. Illustrative presentation components include a display device, speaker, printing component, vibrating component, etc.

I/O ports 1118 allow computing device 1100 to be logically coupled to other devices including I/O components 1120, some of which can be built in. Illustrative components include a microphone, joystick, directional pad, monitor, scanner, printer, wireless device, battery, etc.

Embodiments described herein can be understood more readily by reference to the following Examples. Elements, apparatus, and methods described herein, however, are not limited to any specific embodiment presented in the Examples. It should be recognized that these are merely illustrative of some principles of this disclosure, and are non-limiting. Numerous modifications and adaptations will be readily apparent without departing from the spirit and scope of the disclosure.

### EXAMPLES

### Ultrasound with Line Focus

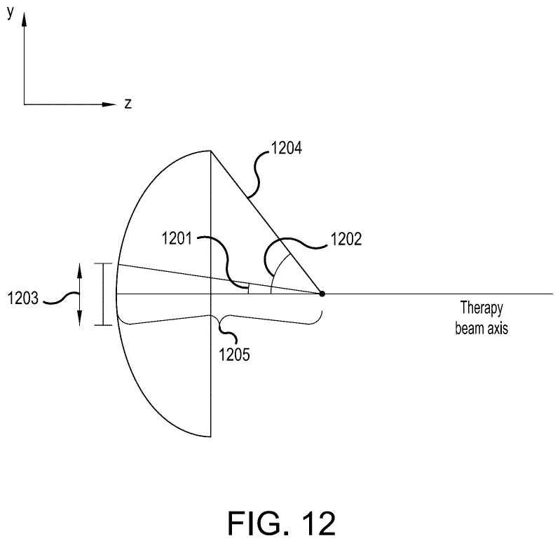

Ultrasound therapy applications, including those described herein, can use a point focus to heat tissue and/or mechanically disrupt tissue. Heating tissue is accomplished by creating a high intensity with sufficient absorption at the focus, and with sufficient time to raise the temperature of tissue. Mechanically disrupting tissue is accomplished by creating a peak negative pressure of sufficient magnitude in the intended target tissue. In some applications, heating is limited or circumvented by keeping ultrasound pulses short and/or with low duty cycles. Further, in many applications, a focused bowl can be used, because every point on the resonating surface is pointed to the same location. This creates exceptional focal gains which are related to the area of the bowl, the focal distance, and the frequency. The focal gain ‘FG’ equation is given by Equation (2):

$${F}_{G} = \frac{A * f}{v * {z}_{f}} , \qquad ( Equation 2 )$$

where ‘A’ is the area of the bowl in meters, ‘f’ is the frequency in Hertz, ‘v’ is the velocity of propagation in meters per second (for instance, 1540 m/sec for water), and ‘zf’ is the focal distance of the bowl (also the radius of curvature). Bowls are useful for focusing the power from a transducer to a point. The area A of the bowl can be calculated from Equation (3):

$$A = 2 * π * {z}_{f}^{2} * ( cos ( {θ}_{1} ) - cos ( {θ}_{2} ) ) , \qquad ( Equation 3 )$$

where θ₂ and θ₁ represent the angles 1202 and 1201 between the beam axis and the bowl edges, as shown in FIG. 12. More specifically, FIG. 12 shows a cross-section of a bowl, including angles 1202 and 1201, which are determined by the inner diameter 1203 and the outer diameter 1204. The focal distance of the bowl (z_{f}) is represented by reference number 1205.

It is further to be understood that other shapes can also be used to generate the necessary pressure to exceed the required cavitational pressure in tissue, and the shape is not particularly limited. For example, cylindrical, spherical, parabolic, or paraboloid shape can also be used. However, a point focus is not as effective at treating large volumes at a fast rate. This has been circumvented (including in the present disclosure) by adding a motion or translation mechanism to enhance the speed of delivery, by using multiple transducers simultaneously, or by electronically focusing the transducer.

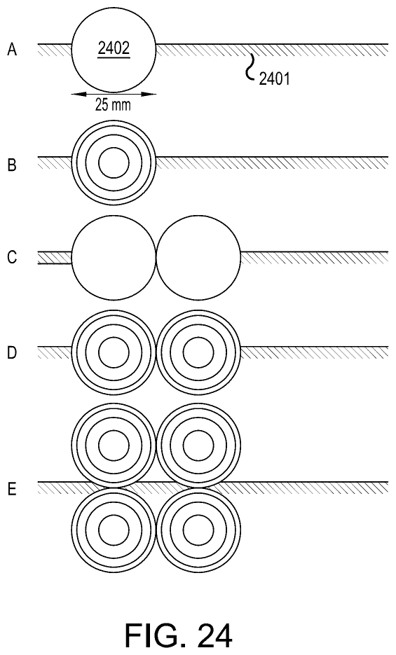

FIG. 24 illustrates embodiments using multiple transducers simultaneously. In the embodiments of FIG. 24, movement of transducer 2402 is effected by motion mechanism 2401. In some embodiments, motion mechanism 2401 may be a lead screw, crankshaft, cylindrical cam, cam and follower, quick return mechanism, scotch-yoke, and/or rack and pinion.

In row A of FIG. 24, transducer 2402 is connected to the motion mechanism 2401, and only one region of tissue is treated at a time.

Row B of FIG. 24 shows transducer 2402 as an annular array, which can in some embodiments enable the creation of bubbles at multiple depths. An annular array allows for treatment of various target areas or volumes at different depths in one pass rather than multiple passes. Furthermore, because a duty cycle for histotripsy could be less than 1% in some instances, it is possible to interleave or raster different depths and/or different treatment points in one treatment, further reducing treatment time. In some embodiments, an annular array may have sufficient channels to move a beam a significant distance, such as, for example, +/−3 mm (for a bowl of the same size as conventional designs, and with the same amount of travel). In some instances, time reduction may be increased by reducing therapy time per site. In some embodiments, reductions of up to −75%, up to −70%, up to −65%, up to −60%, up to −50%, or up to −45% can be obtained, wherein the percent is represented as a negative value to denote a reduction (as opposed to an increase) in time. In some embodiments, a reduction of at least −10%, at least −15%, at least −20%, or at least −25% is obtained. In some embodiments, a reduction of −41% for 50 msec per mechanical injury point or MIP is obtained. In some embodiments, the beam may be raster in depth to interleave pulse delivery, while maintaining the same PRF which, in some embodiments, could offer further enhanced time reductions, which are constant for the same therapy time per MIP. In some cases, time reductions of at least −67% can be obtained in embodiments with rastering.

Row C of FIG. 24 illustrates an embodiment wherein two transducers 2402 are placed together. This embodiment also allows for a reduction in treatment time, since twice the distance is covered for each line delivery in half the amount of time. As such, the embodiment of Row C can achieve reductions in treatment time of −50%.

Row D of FIG. 24 illustrates an embodiment wherein the techniques shown in Row B and Row C are combined. Specifically, in Row D, two therapy annular array transducers are placed together, such that twice the distance is covered for each line delivery in half the amount of time. Further reductions in treatment time can be obtained with this embodiment. For instance, in cases wherein each depth is treated sequentially, time savings of −71% can be obtained. As another example, in cases wherein the device uses a rastering approach, since the duty cycle is only 1%, time reductions of −83% may be obtained.

Row E of FIG. 24 illustrates an embodiment wherein four therapy annular array transducers are placed together. In such embodiment, four times the distance is covered for each line delivery in a quarter of the time, thereby reducing the treatment time even further. For instance, in cases wherein each depth is treated sequentially, time savings or reductions of −85% may be obtained. As another example, in cases wherein the device uses a rastering approach, time reductions of −92% may be achieved.

In other embodiments, rather than applying a motion or translation mechanism, it is also possible to use a line focus to enhance the delivery rate. Three different example designs that generate a line focus are described further below: (i) cylinder segment, (ii) flat plate with lens, and (iii) flat plate with a reflector. A line focus at a depth of at least 1.5 mm can be obtained, including using transducer specifications in Table 3 below. In some embodiments, the focal gain may be further increased by focusing along the line dimension, which may further increase the possibility of achieving a target pressure for cavitation.

TABLE 3
Transducer specifications

| SPECIFICATION | DESCRIPTION |
|---|---|
| Operating Frequency | 10 MHz +/− 1 MHz |
| Operating Bandwidth | >1.5 MHz or 15% |
| Therapy Line, Length | 9.5 mm +/− 0.5 mm |
| Therapy Line, Width | <0.3 mm (focused dimension) |
| Therapy Line, Height | <0.6 mm (beam axis, depth) |
| Focal Depth in Tissue | 1.5 mm +/− 0.5 mm |
| Maximum Acoustic | 200 W/cm² |
| Operating Intensity | |
| Average Acoustic | 50 W/cm² |
| Operating Intensity | |
| Acoustic Efficiency | >65% when measured at the transducer |
| Maximum On-time | 1 second |
| Width | <4.5 mm (orthogonal to line, focus dimension) |
| Maximum Length | 10.0 mm +/− 0.5 mm |
| Nominal Focal Gain | 5 < F_{G} |
| along the Line | |

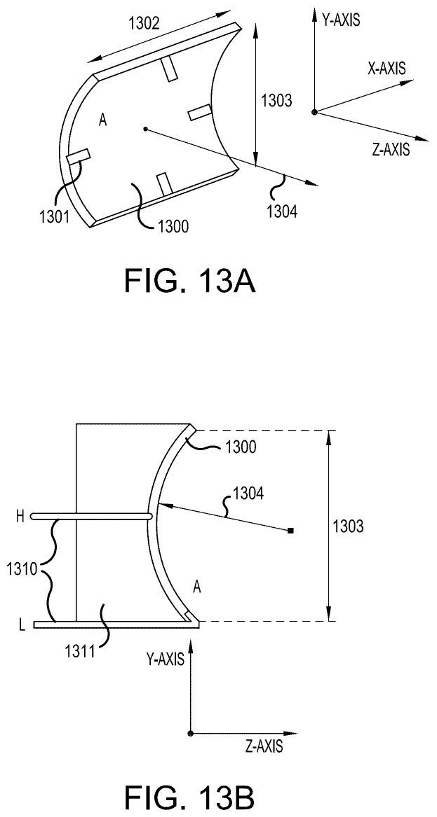

FIGS. 13A and 13B illustrate an exemplary cylinder segment 1300 which operates at 10 MHz. FIG. 13A illustrates a 3D perspective view, and FIG. 13B illustrates a cross-section of the cylinder segment 1300. Cylinder segment 1300 may be, in some embodiments, a piezoelectric cylinder, such as a cylinder made from a piezoelectric ceramic material (e.g., lead zirconate titanate or “PZT”). The cylinder has a length 1302, a height 1303, and a radius 1304. The patient facing side A (concave) can be electrically attached to an electrical connection 1301. In some embodiments, electrical connection 1301 may be a ground electrode. Multiple electrical connections 1301 may be employed so as to lower the return resistance. The convex side B of cylinder segment 1300 is shown in FIG. 13B, and can be electrically attached to wires 1310. Wires 1310 could be coax cables, twisted pairs, etc. The concave side B may have a low impedance backing 1311. As shown in FIG. 13B, wires 1310 may attach to a high side H and a low side L of an excitation form (not shown).

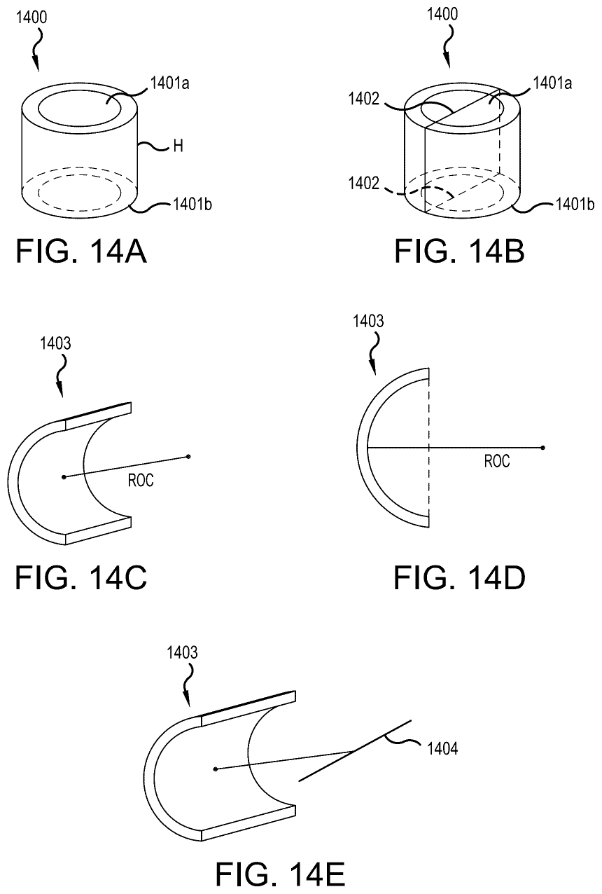

FIG. 14A shows a sketch of a cylinder shell 1400. The cylinder shell has a base 1401a, base 1401b, and a height H. The thickness of the cylinder is selected based on the frequency of operation. FIG. 14B shows chords 1402 on the top and bottom bases 1401a and 1401b (respectively) of the cylinder, which define a cut plane that creates a cylinder shell segment 1403 (shown in FIGS. 14C and 14D). Two chords 1402 are on the same points on the top and bottom bases 1401a and 1401b, respectively, of the cylinder. As shown in FIGS. 14C and 14D, the cylinder shell segment 1403 has a uniform radius of curvature ROC that focuses in only one dimension, and thus creates a line focus 1404 (see FIG. 14E). If the focal gain is sufficient from the cylinder surface to the focus, then tissue heating can be localized along the line if the surface intensity of the segment is sufficiently high and the dose duration is long enough. Furthermore, sufficient focal gain and intensity may enable cavitation along a line. Specifically, an increase in the focal gain can be achieved, in some embodiments, through focusing along the dimension of the line. In some instances, such approach may reduce the line length for a given transducer size, but such can be addressed by increasing the overall size of the transducer along the line dimension.

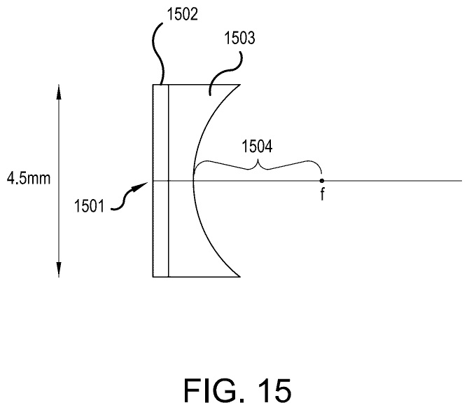

An exemplary flat plate with lens is illustrated in FIG. 15. Specifically, FIG. 15 shows a cross-section of a flat plate 1501 formed from a piezoelectric ceramic material that operates around 10 MHz bonded via bond 1502 to a lens 1503. In the embodiment of FIG. 15, the piezoelectric ceramic material is PZT. However, it is to be understood that any material which exhibits piezoelectric behavior may be employed, including for example, PZT, piezoelectric micromachined ultrasound transducers (PMUTs), and capacitive micromachined ultrasound transducers (CMUTs). One method of creating a line focus is using a composite PZT with an aluminum lens that matches the composite acoustic impedance. In the non-limiting design of this Example, the flat plate measures 10 mm by 4.5 mm (45 mm² area) and has a radius of curvature to focus (1504) at 3.6 mm relative to the concave surface of the lens. The focal gain will be slightly less than the cylinder segment because the area has been reduced and the aluminum lens (if utilized) creates standing waves that yield a non-uniform intensity on the lens surface. Ground electrodes (not shown) are attached on the patient facing side of the transducer and an excitation electrode is soldered to the other side. The excitation along with the ground return are brought together in a coaxial cable. A housing is again used to seal the back of the transducer and prevent water ingress. In some preferred embodiments, the lens has a surface roughness of 0.2 μm or less (Ra, roughness average). The maximum acoustic power of the exemplary flat plate is 90 W.

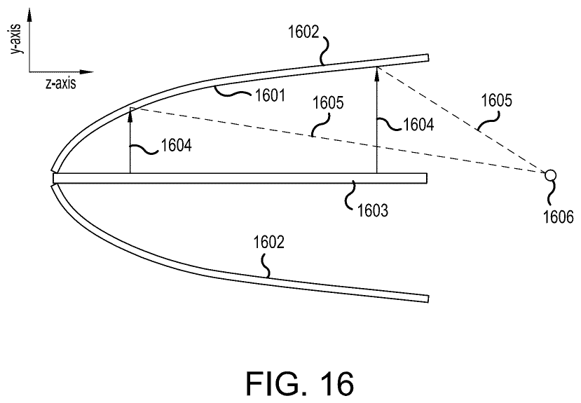

Another approach for achieving a line focus is to use a flat (uncurved or substantially uncurved) plate and a reflector, such as a parabolic reflector. In this design, a flat piezoelectric ceramic plate (e.g., a PZT plate) is sandwiched between two reflectors. Although ultrasound diffracts, the design assumes that the PZT plate and reflector are sufficiently close together to eliminate the diffraction concern. FIG. 16 shows a side or sectional view of the PZT plate sandwiched or disposed between two acoustic reflectors.

In FIG. 16, a reflective surface f(z) (reference number 1601) is provided by reflectors 1602. The reflective surface 1601 can have a geometry defined as described below. In some embodiments, the curvature of the top and bottom reflectors is governed or defined by Equation 4:

$$y = f ( z ) = \pm ( \frac{- 1}{2 * {z}_{f}} {z}^{2} + z ) . \qquad ( Equation 4 )$$

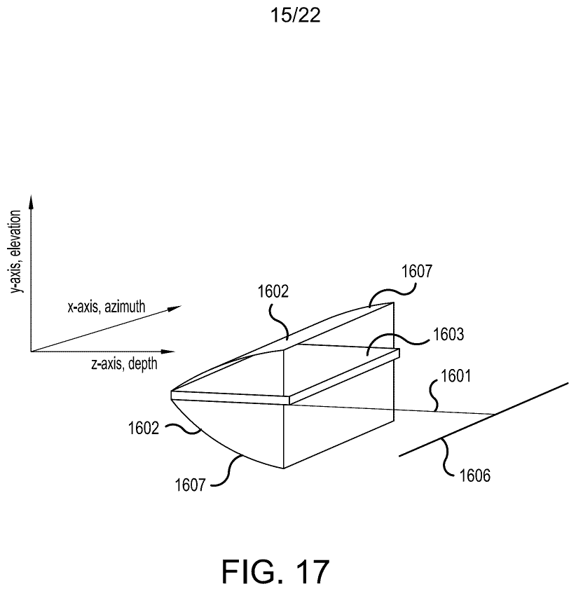

In the specific embodiment illustrated in FIG. 16, the PZT plate 1603 is 10 mm wide by 3 mm in length with a thickness that yields a resonance of 10 MHz. In the embodiment of FIG. 16, both sides of the PZT plate 1603 are surrounded by water or a material with acoustic impedance similar to water. Incident waves 1604 are reflected on reflector 1602, and the reflected waves (1605) create the line focus 1606. Electrodes (not shown) are used on both the bottom and top of the PZT plate 1603, and the design is indifferent to the location of the excitation and ground electrodes. One possible connection scheme is to have part of the PZT plate 1603 outside of the water environment (e.g., where reflectors intersect) as a location to attach the electrical leads. This eliminates or minimizes possible interference of the solder with the plate vibration. If the reflector 1602 is metal, part of the PZT plate 1603 could be parylene-coated (or coated with another electrically insulating material) to reduce the chance of shorting the electrodes. A housing can also be used to isolate the electrical connections. In some preferred embodiments, the reflectors 1602 have a surface roughness of 0.2 μm or less (Ra, roughness average). Moreover, in some preferred embodiments, the maximum translation or tilt of the transducer plate 1603 relative to ideal position to the reflector 1602 is 10 μm or 0.2 degrees, respectively. Maintaining such small ranges can be helpful for avoiding amplification of misalignment to the acoustic performance. Because there is a top and bottom reflectors 1602, misalignment affects the constructive interference pattern at the focus 1606. The total active surface area for the specific embodiment of FIG. 16 is 60 mm² since there is a top and bottom surface. The maximum power from this exemplary device is 120 W. FIG. 17 illustrates a three-dimensional or 3D perspective view of the device of FIG. 16. FIG. 17 shows the curvature (1607) of the reflectors 1602.

It should further be noted that, though not specifically shown in the drawings, the plate (e.g., the PZT plate) could be patterned, such as by etching, to provide individual elements or apodization. For example, in some cases, the plate (e.g., the PZT plate) can comprise an electrode pattern that shades the material closer to the ultrasound focus of the overall device. Similarly, it is also possible to pattern the reflector(s), for example to create an apodization correction. In addition, in some embodiments of a device described herein, the flat plate (e.g., the PZT plate) is separated into a plurality of individually controlled elements. Such a structure can allow elements closer to the focus to be driven with less power or elements closer to the origin to be driven with more power. In this manner, a line focus having a more uniform intensity can be obtained.

A simulation was carried out using a “flat plate plus reflector” structure. Specifically, the following parameters were used:
1. Flat plate, 3 mm width (z-axis, depth), 10 mm length (x-axis, azimuth)
2. Ideal Reflector (Zr>>Zi), so reflection coefficient=1 even at different angles

$$R = ( \frac{{Z}_{2}}{cos {θ}_{t}} - \frac{{Z}_{1}}{cos {θ}_{i}} ) / ( \frac{{Z}_{2}}{cos {θ}_{t}} + \frac{{Z}_{1}}{cos {θ}_{i}} ) , \qquad ( Equation 5 )$$

where R is the reflection coefficient of pressure, Z₂ is the reflector acoustic impedance (i.e. steel), Z₁ is the incident medium acoustic impedance (i.e. water), θₜ is the reflector transmission angle governed by Snell's Law, and θᵢ is the incident angle of the wave in the incident medium to the reflector surface. These angles will vary across the reflector.
3. Reflector size, 3.5 mm width (z-axis, depth), 11 mm length (x-axis, azimuth)
4. Intensity at transducer surface, 1 W/cm² both sides of piezoceramic
5. No attenuation
6. Focal depth is 5 mm, due to reflector size, focal depth from exit plane is 1.5 mm
7. Operating frequency is 10 MHz

The results of the simulation were as follows:

$$1. Max Focal Gain = 10.42$$

$$2. Focal Gain @ ( 0 , 0 , 5 ) = 9.32$$

$$3. Total Area ( PiezoCeramic ) = 30 {mm}^{2} ( two sides , 60 {mm}^{2} )$$

4. Line @ 5 mm depth

$$5. Beamwidth z , - 3 dB = 0.377 mm$$

$$6. Beamwidth x , - 3 dB = 9.3 mm$$

$$7. Beamwidth y , - 3 dB < 0.1 mm$$

$$8. Parabolic Reflector Area = 46.39 {mm}^{2} ( two sides , 92.77 {mm}^{2} )$$

9. For seven cylinders, total length is approximately 31.85 mm with additional length for reflector thickness. Height of one is 4.55 mm. Number of cylinders and reflector height can be adjusted for footprint.
10. Line length can be adjusted based on footprint.
11. Cavitation

TABLE 4
Cavitation Thresholds for Various Tissues (data
obtained from Vlaisavljevich (see also Table 1))

| | | PRF = 100 Hz, | | PRF = 1000 Hz, | Young |
|---|---|---|---|---|---|
| | Threshold | 0.05% duty | Threshold | 0.5% duty | Modulus |
| | (MPa) | cycle | (MPa) | cycle | (MPa) |
| Tissue | Nominal | Variance | Nominal | Variance | Nominal |
| Lung | 1.58 | 0.89 | 13.42 | 1.08 | 0.0026 |
| Fat | 17.13 | 1.41 | 13.26 | 1.85 | 0.0032 |
| Kidney | 17.84 | 1.48 | 14.56 | 0.95 | 0.0061 |
| Liver | 19.97 | 0.77 | 17.75 | 1.07 | 0.0087 |
| Heart | 20.03 | 0.36 | 17.06 | 1.28 | 0.0042 |
| Muscle | 21.01 | 0.48 | 19.12 | 0.57 | 0.0062 |
| Skin | 25.10 | 0.69 | 23.21 | 1.01 | 0.014 |
| Tongue | 26.54 | 0.88 | 24.27 | 0.44 | 0.025 |
| Tendon | 26.41 | 0.52 | 24.47 | 0.49 | 380 |
| Cartilage | no cloud | no cloud | 27.28 | 0.85 | 0.9 |
| Bone | no cloud | no cloud | no cloud | no cloud | 18.6 |

Assuming focal gain of 9 and maximum surface intensity of 250 W/cm², then the maximum intensity is approximately 20 kW/cm². This equates to a negative pressure of 24.5 MPa, which is at or above the threshold pressure for multiple tissues. In some embodiments, higher negative pressures may be achieved by focusing in the line dimension and/or using multiple transducers in a tissue acquisition system, directed to the same tissue depth.

At 250 W/cm², this equates to an acoustic power of 150 W per plate or 75 W per side. If a 60% electric to acoustic efficiency is assumed, the this is 250 W of electrical per plate or 150 W per side. If there are seven plates, then this is 1,750 W total instantaneous power. If the duty cycle is 0.5%, then the average electrical power is 8.75 W. Given the efficiency of 60%, the total electrical loss is 3.5 W. It should further be noted that, in some embodiments, cooling of the plates may be used.

In addition, this analysis shows the possibility of exceeding cavitation thresholds using a line focus where the focus only exists in two dimensions (e.g., x-z or y-z). Since the calculations show that this is right at the threshold for most tissue, another method to further improve the focal gain and also maintain the line length is the use of a compound focus. FIG. 17 shows that the energy is only focused in elevation. If the reflectors or aluminum lens offered a slight focusing in the azimuth dimension, then the focal gain could be increased to further exceed the cavitation threshold. This could be accomplished by placing a focus at a slightly deeper depth than the intended line focus in the azimuth dimension. Although the line length may slightly decrease, this could be circumvented by increasing the plate length.

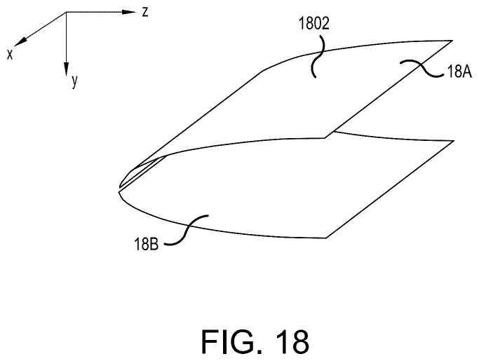

Specific devices for providing line focus (and, in some cases, point focus) are described and illustrated in more detail in FIGS. 18-23. These figures are not necessarily drawn to scale.

FIG. 18 shows a three-dimensional (3D) perspective view of an acoustic reflector 1802 according to one embodiment described herein. The reflector 1802 is one component of the device, and other possible components are described further below. The reflector may only focus in the yz plane, or it may be desirable or beneficial in some instances to have a slight focus in the xz plane, which can improve focal gain. In the embodiment of FIG. 18, the two reflecting surfaces 18 A and 18 B are mirror images of each other (mirrored about the z-axis). Additionally, as illustrated in FIG. 18, the reflector comprises a top reflecting surface or component 18 A, and a bottom reflecting surface or component 18 B. The top and bottom surfaces are in facing opposition to one another. The top and bottom surfaces connect, touch, or have a relatively small separation distance at a first end of the reflector 1802 (on the left of FIG. 18). At a second end (on the right side of FIG. 18), the top and bottom surfaces do not connect or touch, and have a relatively large separation distance. As illustrated in FIG. 18, the top and bottom surfaces 18 A and 18 B define a parabolic or parabola-like reflector having an interior volume in the ‘middle’ of the ‘V’ or ‘U’ of the parabola or parabola-like shape. This volume can be a receiving space for additional components, as described further below. Additionally, in some preferred embodiments, the top and bottom surfaces are symmetric or substantially symmetric and have complementary or similar shapes and sizes in one, two, or three dimensions.

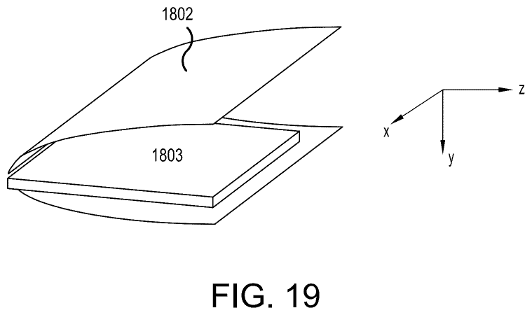

As illustrated in FIG. 19, a piezoelectric plate 1803 has been added to the middle of the acoustic reflector 1802 illustrated in FIG. 18. Moreover, in the embodiment of FIG. 19, the distances between the plate 1803 and the top and bottom portions/surfaces of the reflector (18 A and 18 B, illustrated in FIG. 18) are the same (e.g., when drawing a line normal to the top surface of the plate toward the top reflector component, and when drawing a line normal to the bottom surface of the plate toward the bottom reflector component). The piezoelectric plate 1903 can be formed from a piezoelectric material, including a piezoelectric material described herein. Additionally, in some embodiments, the reflector 1802 can comprise slots, grooves, or other fasteners or retaining means on the two ends in the yz planes, to assist with the alignment of the piezoelectric plate 1803. In some preferred embodiments, the reflector surface is slightly larger than the piezoelectric plate 1803 in the x and z dimensions (e.g., up to 10% or up to 20% larger).

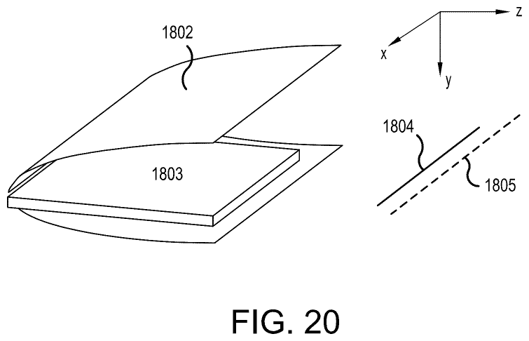

FIG. 20 illustrates a therapy line 1805 which has been focused in y and z dimensions, using the system or device illustrated in FIGS. 18 and 19. The effective length of therapy line 1805 is approximately equal to the width of the plate 1803 in the x dimension. As mentioned previously, a slight focus in x and z dimensions may be added to help improve the overall focal gain. In this case, the therapy line length 1804 would slightly decrease for an identical plate width and possibly move to a shallower depth. Not intending to be bound by theory, it is believed that this design can harness or use all or substantially all of the acoustic energy generated by the top and bottom surfaces of the plate 1803. In addition, in this embodiment, there is no backing, which means heating of the backing does not occur, and more energy propagates into the patient. Moreover, if the piezoelectric plate 1803 and reflector 1802 are in water or water cooled, then the configuration of the device of FIGS. 19-20 allows for better removal of thermal energy at the piezoelectric plate 1803, as compared to some other configurations. Heat may be removed from both sides rather than just one side.

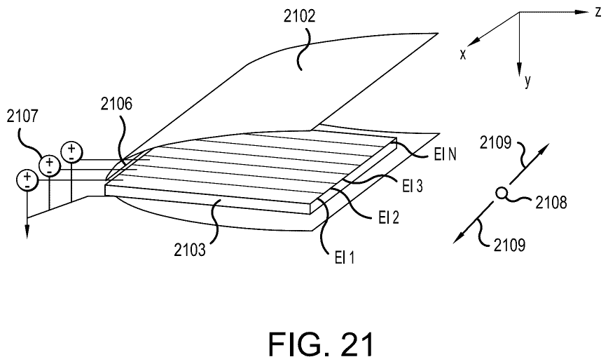

FIG. 21 illustrates a perspective view (not necessarily to scale) of a more specific device according to one embodiment described herein. The device of FIG. 21 comprises a curved, symmetric reflector structure 2102 similar to that described above and illustrated in FIGS. 18-20. In FIG. 21, the piezoelectric plate 2103 (which is disposed in the middle of the receiving space of the reflector 2102, similar to FIGS. 19 and 20) is subdivided into multiple, independently controlled elements El 1, El 2, El 3, . . . . El N) in the x-dimension. These elements can be formed from the same or different piezoelectric materials. This structure can be described as a linear array. However, in this embodiment, the front and back of the piezoelectric plate 2103 are communicating acoustic energy to the field. Electrical connection to the array may also be simplified since part of the plate 2103 (which may be a 2-2 composite, for instance) can “hang” outside of the reflector (e.g., in area 2106, out the “back” or left hand side of the reflector in FIG. 21, such that the top and bottom components, portions, or surfaces of the reflector 2102 “clamp down” on the plate 2103 at the back, or the plate 2103 is disposed between the top and bottom components, portions, or surfaces where they would otherwise touch or come into contact, or be closer to one another). In some embodiments, one side of the plate (e.g., in the x-dimension) gets a signal connection and the other side of the plate gets a ground connection. By having separate control of the delays in the x dimension via independently controlled element 2107, a line focus 2108 or point focus 2109 may be created. Furthermore, a point focus may be swept or restored back and forth electronically, as illustrated by the “spot” and double arrows in FIG. 21.

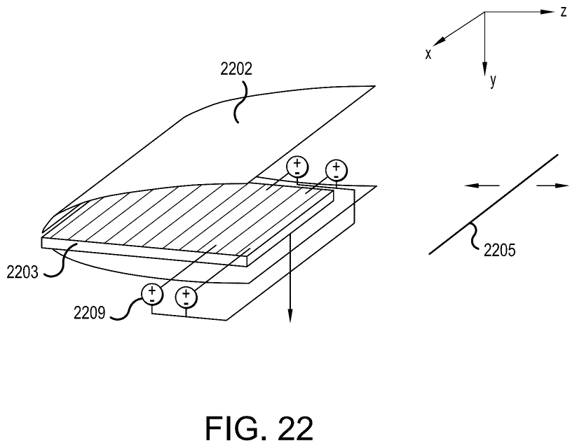

Another embodiment is illustrated in FIG. 22. The device of FIG. 22 comprises a curved, symmetric reflector structure 2202 similar to that described above and illustrated in FIGS. 18-20. However, in the device of FIG. 22, the piezoelectric plate 2203 is subdivided into multiple, independently controlled elements 2209 in the z-dimension. Because there remains no or only slight focusing in the x-dimension, the therapy line 2205 can be moved in the z-dimension as well as split. Again, the front and back of the piezoelectric plate 2203 are communicating acoustic energy to the field. Electrical connection to the array may also be simplified, since part of the plate 2203 (which may be a 2-2 composite in some embodiments) can hang outside of the reflector 2202, as described above regarding FIG. 21. In some cases, one side of the plate 2203 (e.g., in the z-dimension) gets a signal connection and the other side of the plate gets a ground connection. By having separate control of the delays in the z dimension, the effects of the reflector 2202 may be modified to either move the line focus in or out. This can be advantageous for enabling therapy at multiple depths depending on the patient and location of the tissue type to be affected. It is also possible with the time delays to split the focus such that the focus is not at y=0. If the top and bottom part of the plate 2203 are addressed separately (independent time delays, top and bottom), then the beam can be focused along the y-axis.

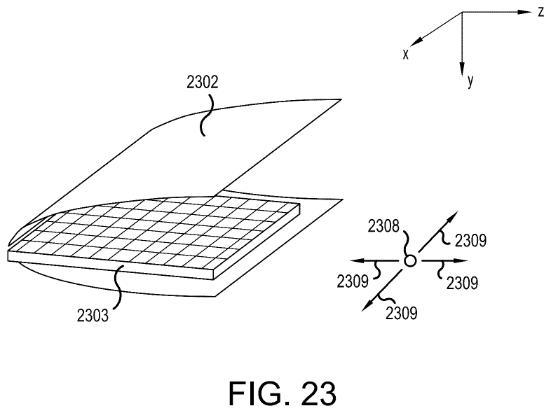

FIG. 23 illustrates yet another exemplary embodiment of a device for providing a line focus 2309 or a point focus 2308. In the embodiment of FIG. 23, a piezoelectric plate 2303 in the form of a row-column transducer has been added to the reflector 2302. In some cases, the row-column transducer 2303 comprises or consists of a 1-3 composite material with edge connections. Such a structure allows the acoustic energy to be focused at different depths as well as in the x-dimension. The focus could consist of a line focus 2309 or point focus 2308, where the point focus 2308 may be restored or swept back and forth electronically, as described further herein.

Some additional exemplary, non-limiting embodiments of methods, systems, and computer storage media are provided below.

Embodiment 1. A method of treating a region of biological tissue of a patient in need thereof, the method comprising: positioning an ultrasound pressure wave in a target volume of the region of biological tissue to form a cavitation bubble cloud in the target volume; and maintaining the cavitation bubble cloud in the target volume for a time period sufficient to produce homogenate within the target volume, wherein the homogenate is fractionated within non-homogenized biological tissue in the region of biological tissue.

Embodiment 2. The method of Embodiment 1, wherein the pressure wave is a composite pressure wave comprising a high frequency component wave and a low frequency component wave.

Embodiment 3. The method of Embodiment 2, wherein the high frequency component wave and the low frequency component wave have frequencies in accordance with Equation 1:

$$p = \frac{{A}_{High} - {A}_{Low} sin ( \frac{3 π}{2} + \frac{( 2 n - 1 ) {f}_{Low} π}{2 {f}_{High}} )}{{A}_{High} + {A}_{Low}}$$

where p is a ratio of the magnitude of the last negative peak pressure relative to the maximum negative pressure, n is the number of cycles of the high frequency component wave in one pulse of the ultrasound pressure wave, f_{low} is the frequency of the low frequency component wave, f_{high} is the frequency of the high frequency component wave, A_{Low} is the amplitude of the low frequency excitation, and A_{High} is the amplitude of the high frequency excitation.

Embodiment 4. The method of Embodiments 2 or 3, wherein at least one cycle of the high frequency component wave is positioned in a trough of at least one cycle of the low frequency component wave.

Embodiment 5. The method of any of Embodiments 2-4, wherein one cycle of the high frequency component wave is disposed within each trough of the low frequency component wave.

Embodiment 6. The method of any of Embodiments 2-5, wherein the high frequency component wave is disposed in a compressional part of the low frequency component wave.

Embodiment 7. The method of any of the previous Embodiments, wherein maintaining the cavitation bubble cloud in the target volume delivers a thermal dose to tissue that does not cause denaturation or coagulation within the target volume.

Embodiment 8. The method of any of the previous Embodiments, wherein maintaining the cavitation bubble cloud in the target volume delivers a thermal dose to tissue that causes denaturation or coagulation within the target volume.

Embodiment 9. The method of Embodiment 8, wherein the thermal dose is sufficient to thermally ablate all or substantially all tissue within the target volume.

Embodiment 10. The method of any of the previous Embodiments, further comprising forming a thermal coagulation zone around the target volume.

Embodiment 11. The method of any of the previous Embodiments, wherein positioning the pressure wave in the target volume comprises applying an ultrasound beam to the target volume.

Embodiment 12. The method of Embodiment 11, wherein the ultrasound beam is a pulsed ultrasound beam.

Embodiment 13. The method of Embodiments 11 or 12, wherein a focus of the ultrasound beam is positioned in the target volume.

Embodiment 14. The method of Embodiments 11 to 13, wherein the focus of the ultrasound beam is a point focus.

Embodiment 15. The method of Embodiments 11 to 13, wherein the focus of the ultrasound beam is a line focus.

Embodiment 16. The method of any of the previous Embodiments, further comprising detecting the onset of cavitation, wherein the onset of cavitation is defined as the time t=0 when the cavitation bubble cloud in the target volume begins to form.

Embodiment 17. The method of Embodiment 16, wherein detecting the onset of cavitation is based on acoustic cavitation emissions.

Embodiment 18. The method of Embodiment 17, wherein acoustic cavitation emissions are detected using a detection device.

Embodiment 19. The method of Embodiment 18, wherein the detection device is a transducer.

Embodiment 20. The method of Embodiment 19, wherein the transducer also applies the ultrasound beam to the target volume.

Embodiment 21. The method of any of Embodiments 17-20, wherein the onset of cavitation is detected at the same time as imaging.

Embodiment 22. The method of any of the previous Embodiments, wherein the pressure wave has a peak negative pressure of 10-100 MPa.

Embodiment 23. The method of any of the previous Embodiments, further comprising: positioning the pressure wave in a second target volume of the region of biological tissue to form a second cavitation bubble cloud in the second target volume; and maintaining the second cavitation bubble cloud in the second target volume for a second time period sufficient to produce homogenate within the second target volume, wherein the second target volume differs from the first target volume.

Embodiment 24. The method of any of the previous Embodiments, further comprising: positioning the pressure wave in n additional target volumes of the region of biological tissue to form n additional cavitation bubble clouds in the n additional target volumes; and maintaining the n additional cavitation bubble clouds in the n additional target volume for n additional time periods sufficient to produce homogenate within the n additional target volumes, wherein the n additional target volumes differ from one another and from the first target volume and the second target volume, and wherein n is an integer from 1 to 1,000,000.

Embodiment 25. The method of any of the previous Embodiments, wherein the biological tissue comprises skin tissue, adipose tissue, connective tissue, or muscle tissue.

Embodiment 26. The method of any of the previous Embodiments, wherein the method provides an aesthetic effect.

Embodiment 27. The method of any of the previous Embodiments, wherein the method does not provide a medical or therapeutic effect other than the aesthetic effect.

Embodiment 28. A system for treating a region of biological tissue of a patient in need thereof, the system comprising: one or more ultrasound transducers to provide an ultrasound pressure wave to a target volume in the region of biological tissue and to form a cavitation bubble cloud in the target volume; and a control device to maintain the cavitation bubble cloud in the target volume for a time period sufficient to produce homogenate within the target volume.

Embodiment 29. The system of Embodiment 28, further comprising one or more sources of electromagnetic radiation to provide one or more beams of electromagnetic radiation to the region of biological tissue for imaging, thermal ablation, and/or delivering non-ablating energy to the region of biological tissue.

Embodiment 30. The system of Embodiments 28 or 29, further comprising one or more microneedles to provide treatment to skin.

Embodiment 31. A computer storage medium storing computer-useable instructions that, when used by one or more computing devices, cause the one or more computing devices to treat biological tissue of a patient, the operations comprising the steps of: positioning an ultrasound pressure wave in a target volume of the region of biological tissue to form a cavitation bubble cloud in the target volume; and maintaining the cavitation bubble cloud in the target volume for a time period sufficient to produce homogenate within the target volume, wherein the homogenate is fractionated within non-homogenized biological tissue in the region of biological tissue.

1. A method of treating a region of biological tissue of a patient in need thereof, the method comprising:
positioning an ultrasound pressure wave in a first target volume of the region of biological tissue to form a first cavitation bubble cloud in the first target volume;
maintaining the first cavitation bubble cloud in the first target volume for a time period sufficient to produce homogenate within the first target volume;
positioning the pressure wave in a second target volume of the region of biological tissue to form a second cavitation bubble cloud in the second target volume; and
maintaining the second cavitation bubble cloud in the second target volume for a second time period sufficient to produce homogenate within the second target volume,
wherein the second target volume differs from the first target volume, and
wherein the homogenate is fractionated within non-homogenized biological tissue in the region of biological tissue.

2. The method of claim 1, wherein the pressure wave is a composite pressure wave comprising a high frequency component wave and a low frequency component wave.

3. The method of claim 2, wherein the high frequency component wave and the low frequency component wave have frequencies in accordance with Equation 1:
$$p = \frac{{A}_{High} - {A}_{Low} sin ( \frac{3 π}{2} + \frac{( 2 n - 1 ) {f}_{Low} π}{2 {f}_{High}} )}{{A}_{High} + {A}_{Low}} , \qquad ( Equation 1 )$$
where p is a ratio of the magnitude of the last negative peak pressure relative to the maximum negative pressure, n is the number of cycles of the high frequency component wave in one pulse of the ultrasound pressure wave, f_{low} is the frequency of the low frequency component wave, f_{high} is the frequency of the high frequency component wave, A_{Low} is the amplitude of the low frequency excitation, and A_{High} is the amplitude of the high frequency excitation.

4. The method of claim 2, wherein at least one cycle of the high frequency component wave is positioned in a trough of at least one cycle of the low frequency component wave.

5. The method of claim 2, wherein one cycle of the high frequency component wave is disposed within each trough of the low frequency component wave.

6. The method of claim 2, wherein the high frequency component wave is disposed in a compressional part of the low frequency component wave.

7. The method of claim 1, wherein maintaining the first or second cavitation bubble cloud in the first or second target volume delivers a thermal dose to tissue that does not cause denaturation or coagulation within the first or second target volume, respectively.

8. The method of claim 1, wherein maintaining the cavitation bubble cloud in the first or second target volume delivers a thermal dose to tissue that causes denaturation or coagulation within the first or second target volume, respectively.

9. The method of claim 7, wherein the thermal dose is sufficient to thermally ablate all or substantially all tissue within the first or second target volume.

10. The method of claim 7, further comprising forming a thermal coagulation zone around the first or second target volume.

11. The method of claim 1, wherein positioning the pressure wave in the first target volume comprises applying an ultrasound beam to the first target volume.

12. The method of claim 11, wherein the ultrasound beam is a pulsed ultrasound beam.

13. The method of claim 11, wherein;
a focus of the ultrasound beam is positioned in the first target volume; and
the focus of the ultrasound beam is a line focus.

14. (canceled)

15. (canceled)

16. The method of claim 11, wherein:
the method further comprises detecting an onset of cavitation;
the onset of cavitation is defined as a time t=0 when the cavitation bubble cloud in the first target volume begins to form;
detecting the onset of cavitation is based on a detection of acoustic cavitation emissions;
acoustic cavitation emissions are detected using a transducer;
the transducer also applies the ultrasound beam to the first target volume; and
the onset of cavitation is detected at the same time as imaging.

17-21. (canceled)

22. The method of claim 1, wherein the pressure wave has a peak negative pressure of 10-100 MPa.

23. (canceled)

24. The method of claim 1, further comprising:
positioning the pressure wave in n additional target volumes of the region of biological tissue to form n additional cavitation bubble clouds in the n additional target volumes; and
maintaining the n additional cavitation bubble clouds in the n additional target volume for n additional time periods sufficient to produce homogenate within the n additional target volumes,
wherein the n additional target volumes differ from one another and from the first target volume and the second target volume, and
wherein n is an integer from 1 to 1,000,000.

25. The method of claim 1, wherein the biological tissue comprises skin tissue, adipose tissue, connective tissue, or muscle tissue.

26. The method of claim 1, wherein;
the method provides an aesthetic effect; and
the method does not provide a medical or therapeutic effect other than the aesthetic effect.

27. (canceled)

28. A system for treating a region of biological tissue of a patient in need thereof, the system comprising:
one or more ultrasound transducers to provide an ultrasound pressure wave to a target volume in the region of biological tissue and to form a cavitation bubble cloud in the target volume; and
a control device to:
maintain the cavitation bubble cloud in the target volume for a time period sufficient to produce homogenate within the target volume;
position the pressure wave in a second target volume of the region of biological tissue to form a second cavitation bubble cloud in the second target volume; and
maintaining the second cavitation bubble cloud in the second target volume for a second time period sufficient to produce homogenate within the second target volume,
wherein the second target volume differs from the first target volume.

29. (canceled)

30. (canceled)

31. A computer storage medium storing computer-useable instructions that, when used by one or more computing devices, cause the one or more computing devices to treat biological tissue of a patient, the operations comprising the steps of:
positioning an ultrasound pressure wave in a target volume of the region of biological tissue to form a cavitation bubble cloud in the target volume; and
maintaining the cavitation bubble cloud in the target volume for a time period sufficient to produce homogenate within the target volume;
positioning the pressure wave in a second target volume of the region of biological tissue to form a second cavitation bubble cloud in the second target volume; and
maintaining the second cavitation bubble cloud in the second target volume for a second time period sufficient to produce homogenate within the second target volume,
wherein the second target volume differs from the first target volume,
wherein the homogenate is fractionated within non-homogenized biological tissue in the region of biological tissue.
